(19) United States
(12) Patent Application Publication
(10) Pub. No.: US 2021/0128823 A1
(43) Pub. Date: May 6, 2021

(54) SELECTIVELY MECHANICALLY ACTIVATABLE PREFILLED INFUSION-PUMP DEVICES

(71) Applicant: SteadyMed, Ltd., Rehovot (IL)

(72) Inventors: Assaf Shaked, Tel Aviv (IL); Avi Azoulay, Ashdod (IL); Maya Gartenberg, Tel Aviv (IL)

(73) Assignee: SteadyMed, Ltd., Rehovot (IL)

(21) Appl. No.: 16/638,840

(22) PCT Filed: Aug. 2, 2018
(86) PCT No.: PCT/IL2018/050864

§ 371 Date: Feb. 13, 2020

Related U.S. Application Data
(60) Provisional application No. 62/548,458, filed on Aug. 22, 2017.

(51) Int. Cl. A61M 5/145; A61M 5/158; A61M 5/142

(52) U.S. Cl. CPC A61M 5/1454; A61M 5/158; A61M 5/14248; A61M 2005/1585; A61M 2205/581; A61M 2005/14252; A61M 2005/1583; A61M 2205/587

(57) ABSTRACT

This invention provides a prefilled selectively activatable body-worn infusion-pump assembly for rapid delivery of large volumes or highly viscous volume comprising a housing; a pre-filled aseptically-sealed flexible drug reservoir-containing assembly; a conjoined, coordinately controlled and perpendicularly arranged cannulated needle dispensing and needle insertion assembly operationally connected to and in fluid connection with the pre-filled aseptically-sealed drug reservoir-containing assembly, wherein the cannulated needle dispensing assembly projects generally perpendicularly to a generally planar surface of the housing, promotes insertion of the cannulated needle in skin of a subject and promotes retraction of the needle within the cannulated needle dispensing assembly thereafter, and wherein the needle insertion assembly projects generally in a parallel orientation to a generally planar surface of the housing, which projection initiates opening a fluid path with the pre-filled aseptically-sealed drug reservoir-containing assembly; an engine assembly contained in the housing operationally connected to the pre-filled aseptically-sealed flexible drug reservoir-containing assembly promoting release of a drug contained therein, wherein the engine assembly comprises: a motor; a worm gear, operationally connected to the motor; a lifting gear, operationally connected to the worm gear; a piston operationally connected to the lifting gear; and a chassis fitted with an attachment promoting a floating connection thereto with the worm gear; wherein the worm gear and motor are mounted radially with respect to the chassis; a printed circuit board (PCB) assembly, which mechanically supports, electrically connects and controls the function of at least the engine assembly; and a single step activator that when engaged simultaneously promotes activation of the needle insertion assembly opening a fluid path with the pre-filled aseptically-sealed drug reservoir-containing assembly; activation of the engine assembly; and activation of the cannulated needle dispensing assembly inserting in a skin of a subject.

### BACKGROUND TO THE INVENTION

The treatment of a variety of diseases necessitates repeat or prolonged delivery of an agent by injection and such injections can be performed using specialized injection devices. Such injector devices may deliver relatively large volumes containing the medicament, including volumes of approximately at least one milliliter and including a few milliliters. Injection of such large volumes of medicament may be over the course of many minutes and even up to a few hours. Generally such devices are operated by the patients themselves, although they may also be operated by medical personnel.

Typically, the initiation is effected by the user operating an electrical switch, which causes a controller to operate the device. Operation includes injecting a needle into the user and then causing the injection of medicament into the user's tissue. Biological medicaments are being increasingly developed which comprise higher viscosity injectable liquids and which are to be administered in larger volumes than long-known liquid medicaments.

Particularly in the case of patient-operated devices, which require insertion of a drug cartridge prior to use, the drug delivery process from start to finish can be a complicated multi-step process, including gathering of all of the device components, assembly of the components to produce the device ready for drug administration and sterilization of the injection site before the actual process of injecting the drug can even begin.

For example, the preparation step includes sourcing a sterilizing liquid and a sterilizing swab to apply the sterilizing liquid. The sterilizing liquid then needs to be applied over the intended injection site on a patient's body to ensure the injection site is fully sterilized, and the sterilizing materials then put aside or discarded before a medicament administration procedure can be commenced. Gathering all these materials and performing the sterilizing process is time-consuming and burdensome, and adds complication to the process for the patient. This renders the process intrusive upon his or her daily schedule, and increases the risk that the patient may not correctly perform the drug administration.

There are also limitations as to the maximum volume of liquid medicament one injection site can accept within a predetermined amount of time without causing the patient discomfort, pain, inhibiting pharmacokinetics or causing leakage out of the injection site. To avoid complications of such interactions between the drug and the patient's body, such large-volume biological medicaments should not be administered at the same injection site on the patient's body twice or more in succession. Therefore, this is another factor in the medicament administration process which the patient must take into consideration.

Other aspects of existing designs for cannula insertion devices that are unsuitable include steps that require the user to manually insert the cannula, or do not automatically retract the insertion needles.

It is therefore an aim of the invention to provide a medicament delivery device which is simple to use and helps to reduce the risk of incorrect use by a user and may be more ideal for use with large volumes and viscous materials

### SUMMARY OF THE INVENTION

It is an objective of the present invention to provide a prefilled selectively activatable body-worn infusion-pump assembly for rapid delivery of large volumes or highly viscous volumes.

It is a further objective of the present invention to provide the prefilled selectively activatable body-worn infusion-pump assembly comprising an aseptically pre-filled drug reservoir, a fluid connection means and a cannula insertion means where the assembly can be fitted to the rest of the infusion-pump components while maintaining sterility and requiring no actions on the part of the end user other than removing the cannula protection cap, adhering the assembled device onto the injection site and pressing a button.

It is an objective of the present invention to provide a prefilled selectively activatable body-worn infusion-pump assembly containing a low profile mechanism that upon a triggering action by the user, inserts a subcutaneous cannulated needle, which in some aspects, automatically retracts the needle component in a simple and efficient manner.

It is another objective of the present invention that the operation of a single button performs all drug delivery device operations.

It is another objective of the present invention that in the absence of appropriate orientation of the skin sensor, activator initiation and thereby drug delivery is prevented.

It is another objective of the present invention that the operation of the needle piercing the drug container septum is simultaneously or essentially simultaneously accompanied by subcutaneous insertion of the cannulated needle and pump activation.

It is another objective of the present invention that the operation of a single button performs or promotes operation of the needle piercing the drug container septum, pump activation and cannula insertion mechanism triggering and adaptations of said single button facilitate simultaneous or essentially simultaneous, i.e. rapid sequential activation of the three operation steps so that opening of a fluid path, drug pumping from a reservoir and cannula insertion and drug delivery therethrough are provided in a controlled and regulated manner.

In some aspects, such single button activation mechanism provides for drug dispensing in a simple manner, requiring no further involvement of the patient in promoting drug delivery other than pushing a button.

This invention provides a prefilled selectively activatable infusion-pump assembly comprising:
a housing;
a pre-filled aseptically-sealed flexible drug reservoir-containing assembly;
a conjoined, coordinately controlled and perpendicularly arranged cannulated needle dispensing and needle insertion assembly operationally connected to and in fluid connection with said pre-filled aseptically-sealed drug reservoir-containing assembly,
wherein said cannulated needle dispensing assembly projects generally perpendicularly to a generally planar surface of said housing, promotes insertion of said cannulated needle in skin of a subject and optionally promotes retraction of said needle within said cannulated needle dispensing assembly thereafter, and
wherein said needle insertion assembly projects generally in a parallel orientation to a generally planar surface of said housing, which projection initiates opening a fluid path with said pre-filled aseptically-sealed drug reservoir-containing assembly;
an engine assembly contained in said housing operationally connected to said pre-filled aseptically-sealed flexible drug reservoir-containing assembly promoting release of a drug contained therein, wherein said engine assembly comprises:
   a motor;
   a worm gear, operationally connected to said motor;
   a lifting gear, operationally connected to said worm gear;
   a piston operationally connected to said lifting gear; and
   a chassis fitted with an attachment promoting a floating connection thereto with said worm gear;
      wherein said worm gear and motor are mounted radially with respect to said chassis;
a printed circuit board (PCB) assembly, which mechanically supports, electrically connects and controls the function of at least said engine assembly; and
a single step activator that when engaged simultaneously promotes:
   activation of said needle insertion assembly opening a fluid path with said pre-filled aseptically-sealed drug reservoir-containing assembly;
   activation of said engine assembly; and
   activation of said cannulated needle dispensing assembly inserting in a skin of a subject.

In some aspects, the conjoined, coordinately controlled and perpendicularly arranged cannulated needle dispensing and needle insertion assembly are arranged in an orientation that is substantially perpendicular with respect to each other.

In some embodiments, deployment of the cannulated needle dispensing assembly is controlled by one or more springs operationally attached thereto. In some aspects, such springs are in a compressed state prior to activation, whereupon release of same results in deployment of the cannulated needle dispensing assembly. In some aspects, the needle and cannula components of the dispensing assembly are separately addressable via a first and second spring, respectively, such that needle retraction is regulated individually via the first spring, while cannula insertion is maintained.

In some embodiments, the first spring is separately retractable after deployment of said second spring.

In some embodiments, the prefilled selectively activatable infusion-pump assembly further comprises:
an engine assembly contained in said housing operationally connected to said pre-filled aseptically-sealed flexible drug reservoir-containing assembly promoting release of a drug contained therein, wherein said engine assembly comprises:
a motor;
a worm gear, operationally connected to said motor;
a lifting gear, operationally connected to said worm gear;
a piston operationally connected to said lifting gear; and
a chassis fitted with an attachment promoting a floating connection thereto with said worm gear;
wherein said worm gear and motor are mounted radially with respect to said chassis; and
a printed circuit board (PCB) assembly, which mechanically supports, electrically connects and controls the function of at least said engine assembly;
wherein said single step activator when engaged further simultaneously promotes activation of said engine assembly.

In some embodiments, the single step activator comprises an element engaging an element which promotes closure of a circuit on said PCB assembly thereby activating said engine assembly. In some embodiments, the single step activator comprises a movable element such that said engine assembly is engaged upon deployment of said single step activator and in some embodiments, the single step activator comprises an element engaging an electronic switch activation surface of said engine assembly, powering same.

In some aspects, the PCB assembly operationally controls function of an indicator light or sound relay system, such that the user receives feedback via indicator light or sound when the device is operational, or in some embodiments, when the device is not operational. In some embodiments, additional user instructions may be imparted to the user via use of the indicator light and/or sound relay system.

In some aspects, the single step activator comprises a laterally moving part operationally connected thereto, whose lateral movement removes a blockade of said needle insertion assembly opening a fluid path with said pre-filled aseptically-sealed drug reservoir-containing assembly; said cannulated needle dispensing assembly inserting in a skin of a subject, or a combination thereof.

In some aspects, the single step activator cannot be engaged or deployed as long as a skin sensor device detects that said prefilled selectively activatable infusion-pump assembly is not properly positioned on a skin of a subject.

In some aspects, the needle insertion assembly pierces a septum comprising ribbing on an outer surface of said septum, protrusions around a bore of either face of said septum, or any combination thereof.

This invention also provides a prefilled selectively activatable infusion-pump assembly comprising:
a housing;
a pre-filled aseptically-sealed flexible drug reservoir-containing assembly;
a conjoined, coordinately controlled and perpendicularly arranged cannulated needle dispensing and needle insertion assembly operationally connected to and in fluid connection with said pre-filled aseptically-sealed drug reservoir-containing assembly,
   said cannulated needle dispensing assembly comprising:
      a first needle hub for securing said dispensing needle and associated first spring for deploying and retracting said dispensing needle; and
      a second outer hub for securing said cannula and associated second spring for deploying said cannula;
wherein said second outer hub and first needle hub are in concentric arrangement and said first spring and second spring are separately addressable and retractable within said assembly, and wherein said first spring and said second spring when released from a compressed state propel said needle and said cannula downward, respectively;
   wherein said cannulated needle dispensing assembly projects generally perpendicularly to a generally planar surface of said housing, promotes insertion of said cannulated needle in skin of a subject and promotes retraction of said needle within said cannulated needle dispensing assembly thereafter, and
   wherein said needle insertion assembly projects generally in a parallel orientation to a generally planar surface of said housing, which projection initiates opening a fluid path with said pre-filled aseptically-sealed drug reservoir-containing assembly;
a power source powering drug delivery; and
a single step activator that when engaged simultaneously promotes:
   activation of said needle insertion assembly opening a fluid path with said pre-filled aseptically-sealed drug reservoir-containing assembly; and
   activation of said cannulated needle dispensing assembly inserting in a skin of a subject.

In other embodiments, this invention provides a prefilled selectively activatable infusion-pump assembly comprising:
a housing;
a pre-filled aseptically-sealed flexible drug reservoir-containing assembly;
a cannulated needle dispensing assembly;
a needle insertion assembly operationally connected to and in fluid connection with said pre-filled aseptically-sealed drug reservoir-containing assembly;
an engine assembly contained in said housing operationally connected to said pre-filled aseptically-sealed flexible drug reservoir-containing assembly promoting release of a drug contained therein, wherein said engine assembly comprises:
   a motor;
   a worm gear, operationally connected to said motor;
   a lifting gear, operationally connected to said worm gear;
   a piston operationally connected to said lifting gear; and
   a chassis fitted with an attachment promoting a floating connection thereto with said worm gear;
wherein said worm gear and motor are mounted radially with respect to said chassis; and
a printed circuit board (PCB) assembly, which mechanically supports, electrically connects and controls the function of at least said engine assembly.

In some embodiments, the worm gear comprises a thrust bearing surface disposed of distally from a floating motor shaft connection that is supported by the chassis rather than the motor shaft.

In some embodiments, the assembly further comprises a single step activator that when engaged simultaneously promotes:
activation of said needle insertion assembly opening a fluid path with said pre-filled aseptically-sealed drug reservoir-containing assembly;
activation of said engine assembly; and
activation of said cannulated needle dispensing assembly inserting in a skin of a subject.

In some embodiments, the single step activator comprises a laterally moving part operationally connected thereto, whose lateral movement removes a blockade of said needle insertion assembly opening a fluid path with said pre-filled aseptically-sealed drug reservoir-containing assembly; said cannulated needle dispensing assembly inserting in a skin of a subject, or a combination thereof. In other embodiments, the single step activator comprises a movable element such that said engine assembly is engaged upon deployment of said single step activator. In other embodiments, the single step activator comprises an element engaging an electronic switch activation surface of said engine assembly, powering same. In some embodiments, the single step activator comprises an element engaging an element which promotes closure of a circuit on said PCB assembly thereby activating said engine assembly. In some embodiments, the single step activator cannot be engaged or deployed as long as a skin sensor device detects that said prefilled selectively activatable infusion-pump assembly is not properly positioned on a skin of a subject.

In some embodiments, the cannulated needle dispensing and needle insertion assembly are conjoined, coordinately controlled and perpendicularly arranged. In some embodiments, the cannulated needle dispensing assembly promotes insertion of a cannulated needle into a skin of a subject and promotes retraction of said needle within said cannulated needle dispensing assembly thereafter.

In some embodiments, the cannulated needle dispensing assembly comprises concentric arrangement of a needle hub securing said insertion needle and a bushing separately securing said cannula in said assembly. In some embodiments, the cannulated needle dispensing assembly comprises a first spring, retractable within said assembly, which when released from a compressed state propels said insertion needle downward and a second differentially addressable spring, retractable within said assembly, which when released from a compressed state propels said cannula downward. In some embodiments, the first spring is separately retractable after deployment from said second spring. In some embodiments, the needle insertion assembly pierces a septum comprising ribbing on an outer surface of said septum, protrusions around a bore of either face of said septum, or any combination thereof.

The invention provides a number of prefilled selectively activatable infusion-pump assemblies. The term “selectively activatable” is to be understood to refer to a requirement for an activation step, i.e., a specific action to be taken to produce the outcome. For example, and representing some embodiments, the term “selectively activatable infusion pump assembly” is to be understood to encompass an assembly whose delivery of the drug via known/described mechanisms, is regulated such that an activation step is required or delivery from the infusion pump is prevented.

In some aspects, such activation step is mediated/regulated via the single step activator, which in some aspects is itself subject to regulation via the skin sensor as described herein.

The prefilled selectively activatable infusion-pump assemblies of this invention comprise a housing.

In some embodiments, the housing will be comprised of any suitable material and will be constructed by conventional means, as will be appreciated by the skilled artisan.

In some aspects, the housing may comprise an indicator light, which in some aspects, provides a selective indicator indicating the device being ready for deployment/engagement of the single step activator. In some aspects, the housing may comprise an indicator light that changes color, as a function of whether the device is ready or not for deployment/engagement of the single step activator. In some aspects, the housing may comprise a sound relay system, which in turn provides an audio feedback, serving as an indicator for the device being ready for deployment/engagement of the single step activator. In some aspects, the sound relay system may change the audio indicator emitted, as a function of whether the device is ready or not for deployment/engagement of the single step activator.

In further aspects, the housing may comprise a series of recesses or posts, or other physical buttresses to accommodate and house the various components of the prefilled selectively activatable infusion-pump assembly.

In further aspects, the housing may comprise an observation window, which provides the user with an interior view to provide an indication as to whether the device is deploying/functioning properly.

In some aspects, the housing further comprises at least one basal adhesive panel. In some embodiments, the housing basal surface may be affixed to the skin of a user for deployment, whereby the basal surface comprises at least one panel which contains an adhesive surface, which adheres to the skin of a user. In some aspects, two or more such panels may be incorporated on the basal surface, as needed.

Such adhesive surfaces may be comprised of any suitable adhesive material for affixing the device to the skin of a user, for example as described in European Patent Application Numbers 0413250 or 0092999.

In some aspects, the housing is provided in a water-resistant or splash proof manner, incorporating, for example appropriate gaskets to ensure appropriate sealing. In some aspects, the housing is so constructed to safely incorporate the sterile fluid path components, maintaining sterility of the inner delivery path components.

In another aspect, the housing may further comprise an activation switch, which in some embodiments, is moved laterally, following engagement of the single step activator, which in turn promotes propulsion of the needle insertion assembly opening a fluid path with said pre-filled aseptically-sealed drug reservoir-containing assembly as well as propulsion of the cannulated needle dispensing assembly toward a skin of a subject. In some aspects, the activation switch engagement, for example, via lateral movement, specifically removes a physical blockade previously preventing propulsion of the needle insertion assembly, propulsion of the cannulated needle dispensing assembly, or a combination thereof.

In some aspects, the activation switch may be adapted to comprise an external element for easy implementation, such as a slide switch, or push button, or any other mechanism as will be appreciated, by the skilled artisan.

In some aspects, the “push button” or other externally located relay may comprise a sealing to prevent compromise of sterility of internally located elements in the device, or in some embodiments, the sealing renders the device water-resistant or water proof, or a combination thereof.

In some aspects, the “push button” or other externally located relay may comprise further adaptations or modifications to engage the simultaneous opening of a fluid path with said pre-filled aseptically-sealed drug reservoir-containing assembly, insertion of said cannulated needle in skin of a subject and promoting retraction of said needle within said cannulated needle dispensing assembly thereafter and activation of said engine assembly to promote concerted opening of the fluid path, initiation of drug delivery from the pre-filled aseptically-sealed flexible drug reservoir-containing assembly and piercing of the subject skin to promote delivery thereto.

According to this aspect, and in some embodiments, the “push button” or other externally located relay may comprise further adaptations or modifications to interface with an element of the cannulated needle dispensing and needle insertion assembly, for example, serving as a trigger interface for same.

According to this aspect, and in some embodiments, the “push button” or other externally located relay may comprise further adaptations or modifications to interface with an element of the engine assembly, or in some embodiments, with an electronic switch activation surface, powering same.

According to this aspect, and in some embodiments, the “push button” or other externally located relay may comprise further adaptations or modifications to interface with an element of the skin sensor device, such that when same is moved to the appropriate position, the push button is now freely depressed/engaged.

According to this aspect, and in some embodiments, the “push button” or other externally located relay may comprise further adaptations or modifications, such as, for example specialized guides, which in turn allow coordinated action of the various aspects as described hereinabove.

In some aspects, in reference herein to the single step activator promoting:
activation of said needle insertion assembly opening a fluid path with said pre-filled aseptically-sealed drug reservoir-containing assembly;
activation of said engine assembly; and
activation of said cannulated needle dispensing assembly inserting in a skin of a subject;
   the term “promotes” refers to direct or indirect activation of the indicated parts, for example, via an extension on the activator button that directly interacts with the identified components, or in some embodiments, via an extension on the activator button that in turn otherwise results in the activation of the identified components, for example, via additional relay part components.

In some aspects, the skin sensor prevents accidental operation of the button before the device is placed on the user. In some embodiments, the skin sensor provides a mechanical block for the full deployment/activation of the activator switch, such that unless the sensor is positioned properly on the skin, it is not possible to engage/deploy the activator switch.

In some aspects, the cannula cap may be made of any convenient sterilizable material, and may be further modified to contain a convenient user grip for removal. According to this aspect, and in some embodiments, the cannula cap will be of any appropriate size in terms of length, width, as will be convenient for packaging and ease of manipulation by the user.

In some embodiments, the cannula cap may promote a sealed container maintaining sterility of the cannulated needle dispensing assembly and thereby maintaining sterility of the drug path. In some aspects, the cannula cap may promote inadvertent injury by the user's premature exposure to the cannulated needle dispensing assembly.

In some aspects, this invention provides a pre-filled aseptically-sealed flexible drug reservoir-containing assembly. In some aspects, the pre-filled aseptically-sealed flexible drug reservoir-containing assembly comprises a thin profile reservoir that can hold large volumes of drug solution with less than 10%, or in some embodiments, less than 9%, or in some embodiments, less than 8%, or in some embodiments, less than 7%, or in some embodiments, less than 6%, or in some embodiments, less than 5% residual volume after delivery.

In some aspects, the pre-filled aseptically-sealed flexible drug reservoir-containing assembly comprises a container containing about 5 mL of drug solution, with less than about 0.3 mL residual volume after completion of the delivery cycle.

In some aspects, the pre-filled aseptically-sealed flexible drug reservoir-containing assembly can withstand high pressures and is thereby able to deliver high-viscosity drug formulations in within short time periods. According to this aspect, and in some embodiments, the pre-filled aseptically-sealed flexible drug reservoir-containing assembly can deliver high-viscosity drug formulations within a sterile drug path with linearity and in some embodiments, 10%, or in some embodiments, 9%, or in some embodiments, 8%, or in some embodiments, 7%, or in some embodiments, 6%, or in some embodiments, 5% accuracy with respect to the delivery rate.

According to this aspect, and in some embodiments, the pre-filled aseptically-sealed flexible drug reservoir-containing assembly can deliver high-viscosity drug formulations within a sterile drug path with linearity and in some embodiments, 10%, or in some embodiments, 9%, or in some embodiments, 8%, or in some embodiments, 7%, or in some embodiments, 6%, or in some embodiments, 5% accuracy with respect to the delivery dose.

In some aspects, the pre-filled aseptically-sealed flexible drug reservoir-containing assembly specifically comprises a deeper baseplate and filling channel while maintaining a slim profile housing.

In some aspects the deeper filling channel, in turn provides for the filling nozzle to penetrate through the container during filling and in some embodiments, provides the advantage of reduced foaming of the drug substance and in some embodiments, provides an advantage in terms of faster filling, or both.

According to these aspects, and in some embodiments, the internal volume in the container is larger than 3 ml and a slim profile for same is maintained. In some aspects, the deeper baseplate accommodates increasing the internal volume in the container.

The prefilled selectively activatable infusion-pump assembly will comprise a conjoined, coordinately controlled and substantially perpendicularly arranged cannulated needle dispensing and needle insertion assembly.

In some aspects, this invention provides a conjoined, coordinately controlled and perpendicularly arranged cannulated needle dispensing and needle insertion assembly operationally connected to and in fluid connection with a pre-filled aseptically-sealed drug reservoir-containing assembly, wherein said cannulated needle dispensing assembly projects generally perpendicularly to a generally planar surface of said housing, promotes insertion of a cannulated needle in skin of a subject and promotes retraction of said needle within said cannulated needle dispensing assembly thereafter, and wherein said needle insertion assembly projects generally in a parallel orientation to a generally planar surface of a housing containing same, which projection initiates opening a fluid path with said pre-filled aseptically-sealed drug reservoir-containing assembly.

In some embodiments, the cannulated needle dispensing assembly comprises:
i. a first needle hub for securing said dispensing needle and associated first spring for deploying and retracting said dispensing needle; and
ii. a second outer hub for securing said cannula and associated second spring for deploying said cannula;
   wherein said second outer hub and first needle hub are in concentric arrangement and said first spring and second spring are separately addressable and retractable within said assembly, and wherein said first spring and said second spring when released from a compressed state propel said insertion needle and said cannula downward, respectively.

In some embodiments, the needle insertion assembly propels a needle or any similarly appropriate penetrating structure toward the sealed prefilled drug-reservoir, thereby facilitating penetration of the drug-reservoir conduit opening a fluid path for same. In some embodiments, a specialized septum configured to be between the insertion assembly and sealed prefilled drug-reservoir is pierced by the needle or similarly appropriate penetrating structure as it is propelled therethrough toward the sealed prefilled drug-reservoir.

In some aspects, this invention provides a low profile mechanism, which upon a triggering action by the user, inserts a subcutaneous cannulated needle and then automatically retracts the insertion needle.

In some aspects, the conjoined, coordinately controlled and perpendicularly arranged cannulated needle dispensing and needle insertion assembly relies on a concentric arrangement of a needle hub that secures the insertion needle and an outer hub that secures the cannula. The mechanism operates with two distinct set of springs whose force is tuned to their respective roles. The thus described orientation ensures that both the cannula insertion and insertion needle retraction actions are highly reliable and allows the device to be assembled with both springs in the compressed state, which allows for a low profile mechanism that can fit within the patch pump housing. In some aspects, upon retraction, the insertion needle completes the fluid path connection with the channel in the top of the insertion mechanism housing.

In some aspects the cannulated needle dispensing and needle insertion assembly is a single unit with two functional elements, which are conjoined and coordinately controlled. In some aspects, the term “conjoined” refers to a structural attachment between the two parts, which in some embodiments, is physically associated or in some embodiments, bonded, or in some embodiments, created as a single unit.

The cannulated needle dispensing and needle insertion assembly are arranged in an orientation that is substantially perpendicular with respect to each other.

In some aspects, the cannulated needle dispensing assembly projects generally perpendicularly to a generally planar surface of the housing and promotes insertion of the cannulated needle into skin of a subject and promotes retraction of the needle within the cannulated needle dispensing assembly thereafter.

In some aspects, the needle insertion assembly projects generally in a parallel orientation to a generally planar surface of the housing, which projection initiates opening a fluid path with the pre-filled aseptically-sealed drug reservoir-containing assembly.

In one aspect, the cannulated needle dispensing element comprises a concentric arrangement of a needle hub securing said needle and a bushing separately securing said cannula in said cannulated needle dispensing assembly.

In some aspects, such mechanism operates with two distinct sets of springs whose force is tuned to their respective roles.

Such arrangement ensures that both the cannula and needle insertion and independent needle retraction actions are highly reliable and in some embodiments, allows the device to be assembled with both springs in the compressed state, which in still further aspects, allows for a low profile mechanism that can fit within the low profile housing.

Such arrangement promotes sequential triggering of the sequence for activation of the cannulated needle dispensing element without need for use of additional external triggers to effect same. In some aspects, the fact that the steps are conducted sequentially improves reliability, as one spring is deployed thereby completing its role and only following same is the second spring deployment initiated.

According to this aspect, and in some embodiments, the cannulated needle dispensing assembly comprises a first spring, retractable within the assembly, which when released from a compressed state propels the needle of the cannulated needle dispensing assembly downward and a second differentially addressable spring, retractable within the assembly, when released from a compressed state propels the cannula downward. According to this aspect and in some embodiments, the first spring is separately retractable after deployment from the second spring.

In some aspects, the needle insertion assembly, whose projection initiates opening a fluid path with the pre-filled aseptically-sealed drug reservoir-containing assembly, pierces a septum designed to ensure sterile closure of the pre-filled aseptically-sealed drug reservoir-containing assembly. In some aspects, such septum is designed to deliver high viscosity drug formulations in relatively short periods of time, such that said septum consistently allows for secure and sterile closure of the aseptically filled reservoir, resisting the high pressures generated during operation of the device. In some aspects, such septum is designed to allow for the needle insertion assembly to consistently pierce the septum with relatively low force and without such deflection that it would make contact with the walls of the reservoir or its neck.

In some aspects, such septum is so designed with specially shaped outer and inner faces, to comprise ribs on its outer circumference, and in some embodiments, protrusions around the bore on both faces of such septum and in some embodiments, a combination thereof. According to this aspect, and in some embodiments, such structural modifications minimize insertion force, while at the same time eliminating undesired needle deflection during the fluid connection operation while maintaining ease of making a robust sterile closure of the aseptically filled container.

In some aspects, such control of needle deflection facilitates accurate needle placement and promotes for a more ideally shallow channel in the baseplate of the drug reservoir thus minimizing residual volume.

The prefilled selectively activatable infusion-pump assembly further comprises an engine assembly contained in said housing operationally connected to said pre-filled aseptically-sealed flexible drug reservoir-containing assembly promoting release of a drug contained therein, wherein said engine assembly comprises:
a motor;
a worm gear, operationally connected to the motor;
a lifting gear, operationally connected to the worm gear;
a piston operationally connected to the lifting gear; and
a chassis fitted with an attachment promoting a floating connection thereto with the worm gear;
   wherein the worm gear and motor are mounted radially with respect to the chassis.

It will be appreciated that the term “worm gear” is to be understood as its art-recognized meaning. The term “worm gear” will refer, inter alia, to a mechanical arrangement consisting of a toothed wheel worked by a short revolving cylinder (worm) bearing a screw thread. In some aspects, the worm gear will be understood to comprise a threaded shaft (worm) that mates with a gearwheel (worm wheel) so that rotary motion can be transferred between two shafts at right angles to each other.

In some aspects, the worm gear serves to reduce rotational speed and/or transmit higher torque.

It is a further objective of the invention to provide a prefilled selectively activatable infusion-pump assembly containing a worm gear drive system that transmits high forces to a drive gear from a motor having a planetary gear reduction system, i.e. isolating a sensitive planetary gear from axial and radial forces. The system allows for the easy assembly of an injection molded worm gear that exhibits low friction when transmitting strong loads to a drive gear while isolating the sensitive planetary reduction gear system of the drive motor from strong thrust loads.

In some aspects, the prefilled selectively activatable infusion-pump assembly is so constructed so as to ensure that there is neither axial nor radial load placed on the motor, which in turn ensures that only torque is transferred in engagement of the motor and worm gear.

According to this aspect, and representing embodied aspects of the invention, with regard to the mechanical interfaces within the motor-worm-chassis assembly, in some aspects, there exists a loose connection between the worm gear and the motor, and in some embodiments, there exists a loose connection between the worm gear and the chassis proximal to the motor shaft.

In some aspects, there exists a tight connection between the motor and the chassis elements constraining same. In some aspects, there exists a tight connection between the worm gear and the distal connection to the chassis (i.e. distal to the connection to the point of connection near the motor shaft).

It is an objective of this invention to provide a worm gear having a thrust bearing surface disposed of distally from a floating motor shaft connection that is supported by the chassis rather than the motor shaft. The floating attachment combined with the placement of the thrust bearing surface allows for enough radial displacement of the worm gear during operation as not to transmit damaging radial or longitudinal forces to the motor's planetary gear reduction system while ensuring there is minimal longitudinal play as not to affect the precision of the drive gear rotation. The single thrust bearing surface and floating attachment of the worm to the motor shaft also allows for the simple alignment of the parts during assembly. FIG. 5B depicts some embodiments of this aspect.

The selectively activatable infusion-pump assembly further comprises a lifting gear operationally connected to the worm gear.

In some aspects, the term “lifting gear” refers to any thus designated element that can be used to lift loads. According to this aspect, the lifting gear specifically promotes lifting of the piston, which in turn applies a desired force to the pre-filled aseptically-sealed flexible drug reservoir-containing assembly, to promote controlled delivery of the contents of same to the subject, once the fluid path between the pre-filled aseptically-sealed flexible drug reservoir-containing assembly and conjoined, coordinately controlled and perpendicularly arranged cannulated needle dispensing and needle insertion assembly is opened.

In some aspects, the selectively activatable infusion-pump assembly further comprises a piston operationally connected to the lifting gear, which in turn, as described applies a force to the pre-filled aseptically-sealed flexible drug reservoir-containing assembly, to promote controlled delivery of the contents of same to the subject, once the fluid path between the pre-filled aseptically-sealed flexible drug reservoir-containing assembly and conjoined, coordinately controlled and perpendicularly arranged cannulated needle dispensing and needle insertion assembly is opened.

In some aspects, the selectively activatable infusion-pump assembly further comprises a chassis fitted with an attachment promoting a floating connection with the worm gear wherein the worm gear and motor are mounted radially with respect to the chassis.

In some aspects, the selectively activatable infusion-pump assembly further comprises a printed circuit board (PCB) assembly, which mechanically supports, electrically connects and controls the function of at least the engine assembly.

It is a further object of the invention to ensure that the pre-filled drug-reservoir remains sealed or that a sterile unit containing such drug reservoir be maintained as such, until directly before the activation of the patch-pump, such that the only materials that come in contact with the drug are the plastic and/or glass from which the reservoir is fabricated and one or more septa.

It is a still further object of the invention to open said fluid connection while keeping the operation of said patch-pump as simple as possible and requiring an essentially single activation-step.

According to this aspect, and as referred to herein the term “selectively activatable” is to be understood to refer to a requirement for an activation step, i.e., a specific action to be taken to produce the outcome.

According to this aspect, and in some embodiments, the cannula-containing assembly provides for the delivery of the drug-containing substance liberated from the drug reservoir. In some embodiments, such cannula-containing assembly comprises a part capable of piercing the skin. In some embodiments, such cannula-containing assembly may resemble a venicath or similar structure, which provides for skin puncture to promote subcutaneous delivery. In some embodiments, such catheter-containing part may be flexible or rigid.

In some embodiments, the prefilled selectively activatable infusion-pump assembly comprises a pre-filled aseptically-sealed flexible drug reservoir-containing assembly, which is essentially a thin profile reservoir-containing assembly that can hold large volumes of drug solution with less than 5% residual volume after delivery. For example, and in some embodiments, the drug reservoir-container holds approximately 5 mL of drug solution with less than 0.3 mL residual volume after completion of the delivery cycle. In some aspects, the drug reservoir-container is so constructed so as to withstand high pressure, while facilitating delivery of high-viscosity drug formulations in within short time periods and delivery of same is within a sterile drug path with linearity with respect to the delivery rate.

In some aspects, the invention provides for pre-filled aseptically-sealed flexible drug reservoir containing a deep baseplate, which in some embodiments, is up to 1 mm, or in some embodiments, up to 2 mm, or in some embodiments, up to 3 mm, or in some embodiments, up to 4 mm, or in some embodiments up to 5 mm in depth. In some aspects, the additional clearance does not adversely impact the linearity of drug delivery

In some embodiments, the pre-filled aseptically-sealed flexible drug reservoir is attached, 1-3 mm or in some embodiments, 2-3 mm above the floor of the baseplate, or in some embodiments, such attachment is at a sufficient height above the floor of the baseplate to promote “double-flip” operation of same.

In some aspects, the prefilled selectively activatable infusion-pump assemblies of this invention provide for the ability to prepare an aseptically pre-filled drug reservoir, a fluid connection means and a cannula insertion means where the assembly can be fitted to the rest of the described components while fully maintaining sterility of the assembly and its component parts. In some aspects, there is no action on the part of the end user for initiating delivery in the devices of this invention other than removing the cannula protection cap, adhering the assembled device onto the injection site and pressing a button, while full sterility is maintained.

In some aspects, uniquely the invention provides a means of specific combination of steps that first assembles a sterilized drug reservoir that is aseptically filled, connects same to a pre-sterilized drug path connection system and cannula insertion system and further assembles same within the device housing, to create a functional drug delivery device such that the end user needs only to expose an adhesive surface, remove the cannula cap, adhere the device to the injection site and press a button in order to complete the injection process. Uniquely the invention provides for independent end user obtaining a packaged prefilled selectively activatable infusion-pump assembly of this invention, and independent sole initiation of drug delivery with a single push button device, where no additional medical personnel intervention is required.

Some embodied contemplated devices are explained more fully below, in connection with the figures, but the same shall not be construed as limiting the invention.

All publications, patents, and patent applications mentioned herein are hereby incorporated by reference in their entirety as if each individual publication or patent was specifically and individually indicated to be incorporated by reference. In case of a conflict between the specification and an incorporated reference, the specification shall control. Where number ranges are given in this document, endpoints are included within the range. Furthermore, it is to be understood that unless otherwise indicated or otherwise evident from the context and understanding of one of ordinary skill in the art, values that are expressed as ranges can assume any specific value or sub-range within the stated ranges, optionally including or excluding either or both endpoints, in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

### BRIEF DESCRIPTION OF THE DRAWINGS

FIG. 1 provides an exploded view of certain elements of the prefilled selectively activatable infusion-pump assembly is shown. An embodied top cover 1-20 and bottom cover 1-30 of the housing, indicator light or sound relay 1-40 and observation window 1-50, are shown, as are the embodied skin sensor 1-60, pre-filled aseptically-sealed flexible drug reservoir-containing assembly 1-70, engine assembly 1-80, conjoined, coordinately controlled and perpendicularly arranged cannulated needle dispensing and needle insertion assembly 1-90 and PCB assembly 1-100.

FIG. 2A and FIG. 2B provide basal surface views of an embodied prefilled selectively activatable infusion-pump assembly 2-10 with skin sensor 2-67, cannula cap 2-110, basal panel 2-120, non-adhesive backing peeling tab, 2-130 and PCB assembly 2-100 are shown.

FIG. 3A and FIG. 3B depict top views of an embodied prefilled selectively activatable infusion-pump assembly of this invention. The apical portion of the housing 3-20, indicator light 3-40, observation window 3-50 and activator 3-140, basal panel 3-120, peeling tab, 3-130, apical part of the observation windown placement on top of the pre-filled aseptically-sealed flexible drug reservoir-containing assembly 3-70 and its containment/placement with respect to the chassis 3-180, which is a part of the engine assembly 3-80 are depicted.

FIG. 4A, FIG. 4B, FIG. 4C, FIG. 4D, FIG. 4E and FIG. 4F provide various views of elements of an embodied prefilled selectively activatable infusion-pump assembly of this invention. The outline of the pre-filled aseptically-sealed flexible drug reservoir-containing assembly, primary container 4-70, needle insertion assembly 4-210, with its septum-piercing needle 4-220, conjoined coordinately controlled and perpendicularly arranged cannulated needle dispensing assembly 4-65, cannula cap 4-60, activator button 4-140, which engages and rotates switch 4-230, PCB assembly 4-100, lifting gear -4-190, piston 4-200, motor 4-245, which promotes rotation of the worm gear 4-250, which engages the lifting gear 4-190, which impinges on the flexible pre-filled aseptically-sealed flexible drug reservoir-containing assembly via the piston 4-200, chassis 4-180, attachment for the worm gear 4-250, modified screw interface 4-280 of the lifting gear 4-190, which engages a threaded region 4-290 on the piston, tabs 4-270 on the piston being locked in place in slots 4-260, specialized region on the drive 4-340, appropriately modified interacting components 4-195 on the lifting gear and other elements are shown.

FIG. 5A, FIG. 5B, FIG. 5C and FIG. 5D depict an embodied motor 5-245, worm drive 5-250, chassis 5-360, attachment of the thrust bearing surface 5-300 to the chassis via modified connection 5-390, chassis connection to the motor gear 5-370, slots 5-260 for the accommodation of the tabs on the piston, central pin 5-440, sliding surfaces 5-420 for the accommodation of the worm gear and for the mounting of the motor 5-430 are shown, as well.

FIG. 6A, FIG. 6B, FIG. 6C and FIG. 6D depict certain elements of an embodied prefilled selectively activatable infusion-pump assembly in non-deployed orientation. The chassis 6-180, lifting gear 6-190 and piston 6-200 are shown, as well as piston top surface 6-205, flexible pre-filled aseptically-sealed flexible drug reservoir-containing assembly bounded by an upper wall 6-75 basal surface 6-78 of the flexible pre-filled aseptically-sealed flexible drug reservoir-containing assembly, apical surface 6-205 of the piston, lead screw interface 6-280 of the lifting gear , threaded region 6-290 of the piston, top of the screw interface 6-280 of the lifting gear, top region of the threaded area 6-290 of the piston, and other aspects as described herein are shown.

FIG. 7A, FIG. 7B, FIG. 7C, FIG. 7D, FIG. 7E, FIG. 7F and FIG. 7G depict various views of the elements of the coordinately controlled and perpendicularly arranged cannulated needle dispensing and needle insertion assembly and their operational and fluid connection to the pre-filled aseptically-sealed drug reservoir-containing assembly, including the activator button 7-140, needle insertion assembly 7-210, needle 7-220, trigger 7-230, catheter component 7-710, needle hub 7-530, springs 7-520 and 7-540 and needle hub bushing 7-510 are shown.

FIG. 8A, FIG. 8B, FIG. 8C and FIG. 8D depict additional aspects of the coordinately controlled and perpendicularly arranged cannulated needle dispensing and needle insertion assemblies. The needle insertion assembly 8-90, cannulated needle dispensing assembly 8-220, trigger 8-230, hub and bushing springs 8-510, 8-520 and needle hub 8-530 are shown.

FIG. 9A, FIG. 9B and FIG. 9C depict the regulated release of the cannulated needle dispensing assembly, showing inter alia the trigger 9-230 and needle bushing 9-510 and other aspects.

FIG. 10A, FIG. 10B and FIG. 10C depict another aspect of the cannulated needle dispensing assembly and its bushing, inter alia, depicting the needle 10-500, needle hub 10-530, needle bushing 10-510, needle spring 10-540, slidable surface 10-570, stopper for the needle hub 10-560 and snaps 10-580.

FIG. 11A, FIG. 11B, FIG. 11C, FIG. 11D, FIG. 11E and FIG. 11F describe additional aspects of the needle hub, including inter alia, the needle 11-500 attachment, snaps 11-565, septum 11-600, funnel 11-610 and cannula 11-620.

FIG. 12A, FIG. 12B, FIG. 12C, FIG. 12D and FIG. 12E depict additional embodied insertion needle assembly elements, including, inter alia, insertion needle 12-680, beveled terminus 12-720, sealing element 12-740, connecting neck 12-760, septum sealing ribs 12-790, and terminal modifications 12-800

FIG. 13A, FIG. 13B, FIG. 13C, FIG. 13D, FIG. 13E, FIG. 13F, FIG. 13G, FIG. 13H, FIG. 13I, FIG. 13DJ, FIG. 13K and FIG. 13L depict embodied aspects of the skin sensor and its regulation of device activation. Various embodied aspects are shown, such as activator 13-140, sensor support 13-65, sensor pad 13-67, opto block feature 13-950, spring 13-945, sensor relay system 13-955, opto block feature, also referred to herein as “flag” 13-950, activation button extensions 13-145, 13-144, 13-142, trigger 13-230, insertion needle hub 13-735 and septum 31-215.

FIG. 14A, FIG. 14B, FIG. 14C, FIG. 14D, FIG. 14E, FIG. 14F, FIG. 14G, FIG. 14H and FIG. 14I, schematically depict aspects of an embodied activator and its regulation of drug delivery. Various embodied aspects are shown, such as activator 14-140, push button surface 14-860, sealing structures 14-880, sealing ribs 14-870, locking mechanisms 14-850, stopper 14-847, mounting surfaces 14-890, electronic switch activation surface 14-830 and electronic switch 14-103.

FIG. 15A and FIG. 15B depict a flow chart showing specific combinations of steps that promotes preparation of a sterilized drug reservoir that is aseptically filled, then connected to a pre-sterilized drug path connection system and cannula insertion system and assembled into the functional patch pump device.

### DETAILED DESCRIPTION OF THE INVENTION

This invention provides a prefilled selectively activatable body-worn infusion-pump assembly for rapid delivery of large volumes and/or highly viscous volumes.

In some aspects, the prefilled selectively activatable body-worn infusion-pump assembles of this invention comprise an aseptically pre-filled drug reservoir, a fluid connection means and a cannula insertion means where the assembly can be fitted to the rest of the infusion-pump components while maintaining sterility and requiring no actions on the part of the end user other than removing the cannula protection cap, adhering the assembled device onto the injection site and pressing a button.

In some embodiments, the invention provides a prefilled selectively activatable body-worn infusion-pump assembly containing a low profile mechanism that upon a triggering action by the user, inserts a subcutaneous cannulated needle, which automatically retracts the insertion needle in a simple and efficient manner. In some aspect, the operation of a single button performs all drug delivery device operations.

This invention provides a prefilled selectively activatable infusion-pump assembly comprising:
a housing;
a pre-filled aseptically-sealed flexible drug reservoir-containing assembly;
a conjoined, coordinately controlled and perpendicularly arranged cannulated needle dispensing and needle insertion assembly operationally connected to and in fluid connection with said pre-filled aseptically-sealed drug reservoir-containing assembly,
wherein said cannulated needle dispensing assembly projects generally perpendicularly to a generally planar surface of said housing, promotes insertion of said cannulated needle in skin of a subject and promotes retraction of said needle within said cannulated needle dispensing assembly thereafter, and
wherein said needle insertion assembly projects generally in a parallel orientation to a generally planar surface of said housing, which projection initiates opening a fluid path with said pre-filled aseptically-sealed drug reservoir-containing assembly;
an engine assembly contained in said housing operationally connected to said pre-filled aseptically-sealed flexible drug reservoir-containing assembly promoting release of a drug contained therein, wherein said engine assembly comprises:
   a motor;
   a worm gear, operationally connected to said motor;
   a lifting gear, operationally connected to said worm gear;
   a piston operationally connected to said lifting gear; and
   a chassis fitted with an attachment promoting a floating connection thereto with said worm gear;
      wherein said worm gear and motor are mounted radially with respect to said chassis;
a printed circuit board (PCB) assembly, which mechanically supports, electrically connects and controls the function of at least said engine assembly; and
a single step activator that when engaged simultaneously promotes:
   activation of said needle insertion assembly opening a fluid path with said pre-filled aseptically-sealed drug reservoir-containing assembly;
   activation of said engine assembly; and
   activation of said cannulated needle dispensing assembly inserting in a skin of a subject.

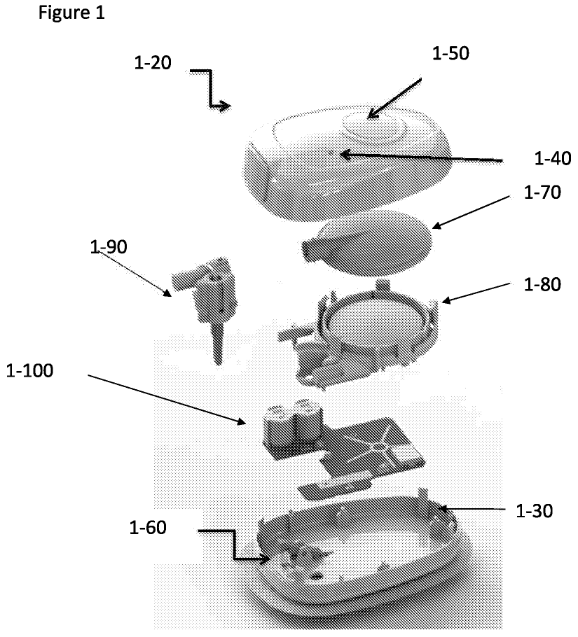

Referring to FIG. 1, an embodiment depicting an exploded view of certain elements of the prefilled selectively activatable infusion-pump assembly is shown. According to this aspect, a top cover 1-20 and bottom cover 1-30 of the housing is seen.

In one aspect, the assembly may comprise an indicator light 1-40 or sound relay, observation window 1-50, elements of which may in some aspects, be observed in the top cover of the housing, and in other aspects on the side or bottom of same. In some aspects, the elements, such as the indicator light or sound relay system, may comprise components on the PCB for regulated activation/signaling via same.

In another aspect, the bottom cover 1-30 provides a top view of a skin sensor 1-60.

As can also be seen in the figure, there is a pre-filled aseptically-sealed flexible drug reservoir-containing assembly 1-70, engine assembly 1-80, conjoined, coordinately controlled and perpendicularly arranged cannulated needle dispensing and needle insertion assembly 1-90 and PCB assembly 1-100.

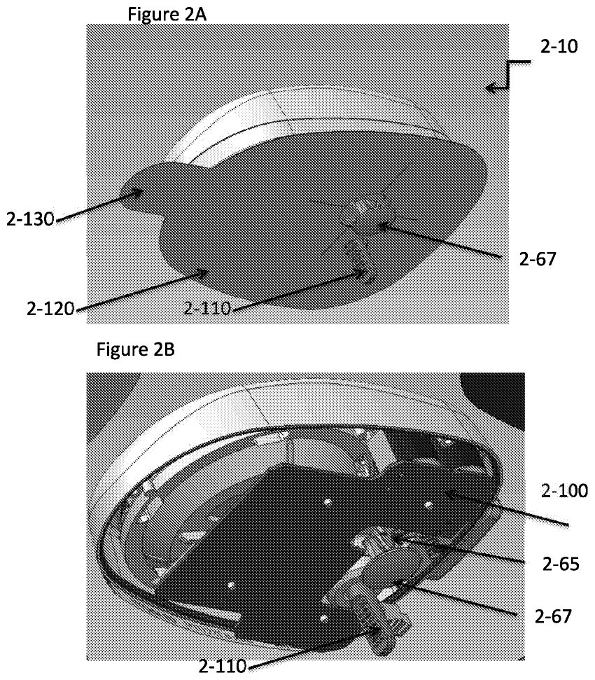

FIG. 2 depicts the basal surface of an embodied prefilled selectively activatable infusion-pump assembly 2-10. In FIG. 2A, the basal view of the skin sensor 2-67 is seen, which contains the sensor surface that abuts the skin of a subject onto which the embodied prefilled selectively activatable infusion-pump assembly 2-10 is placed. Further observed is a cannula cap 2-110, which is a safety cover for the cannulated needle dispensing assembly, which projects generally perpendicularly to a generally planar surface of said housing, as is evident in the figure. Further evident in the figure is a basal panel 2-120, which in this case, essentially covers most of the basal surface. The basal panel 2-120 may comprise an underlying adhesive surface, which is exposed by peeling off a non-adhesive backing covering same, to ensure that the adhesive panel is only exposed at the time affixing the prefilled selectively activatable infusion-pump assembly to the subject is desired. In accordance with this aspect, such adhesive surface is exposed by peeling the non-adhesive backing via the aid of a peeling tab, 2-130.

FIG. 2B shows a basal view with the basal surface of the housing removed, whereby the catheter cap 2-110 and bottom surface of the skin sensor 2-67 is more readily viewed and its orientation and accommodation within the device, for example, vis a vis the PCB assembly 2-100. Connection of the bottom surface of the skin sensor 2-67 to other components of the sensor assembly, e.g. 2-65 is partially shown, as well.

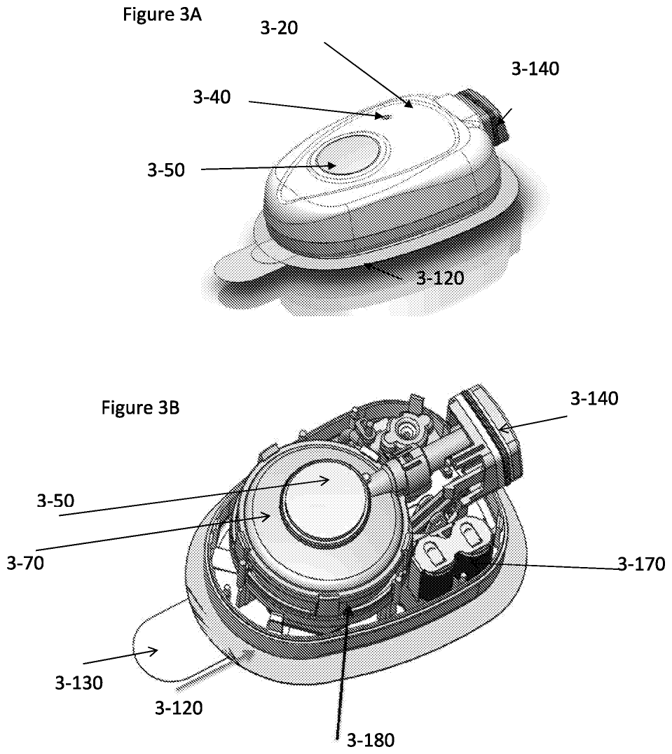

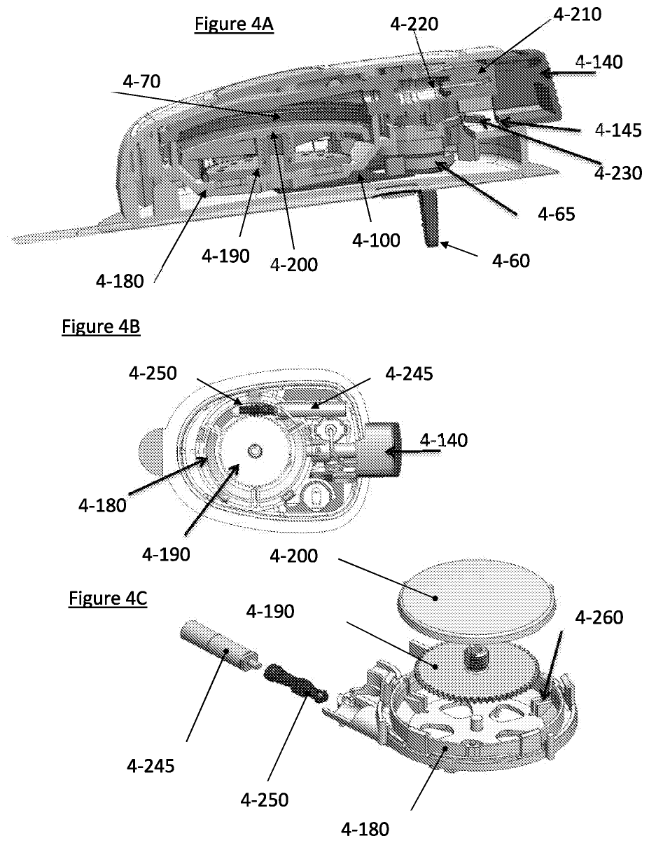

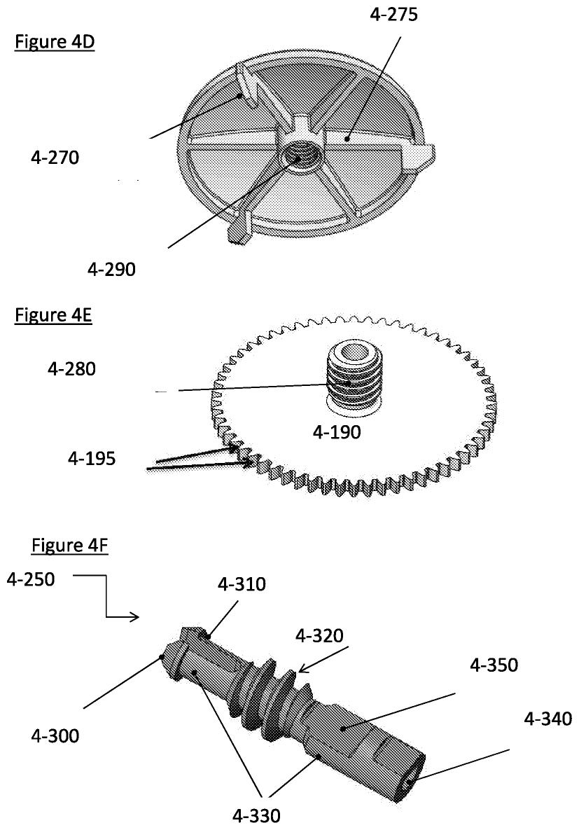

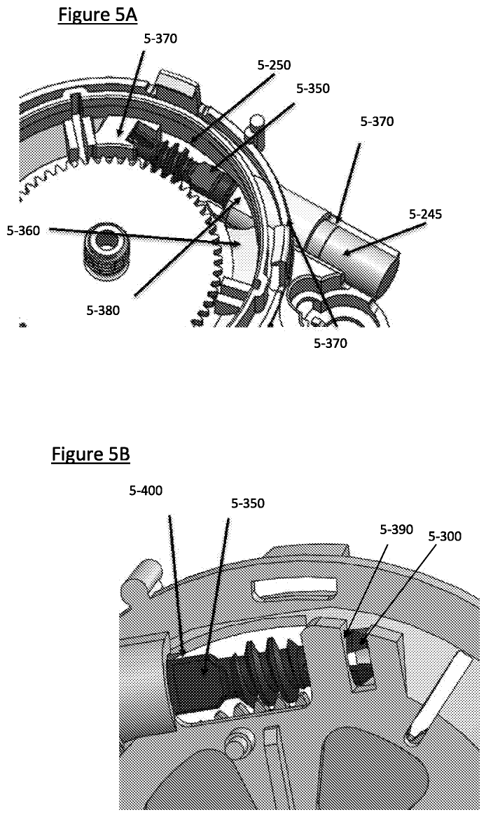

FIG. 3A depicts a top view of an embodied prefilled selectively activatable infusion-pump assembly of this invention. The apical portion of the housing 3-20 is shown, and indicator light 3-40, observation window 3-50 and activator 3-140 are seen. The basal panel 3-120 is shown along with peeling tab, 3-130. Similar to FIG. 2B, FIG. 3B shows an apical view with the apical surface of the housing removed, whereby the orientation of parts is more readily seen. The orientation of the activator 3-140 is shown, as is the apical part of the observation window placement on top of the pre-filled aseptically-sealed flexible drug reservoir-containing assembly 3-70 and its containment/placement with respect to the chassis 3-180, which is a part of the engine assembly. In this aspect, the battery 3-170 is shown, which powers the PCB assembly (not seen in this aspect).

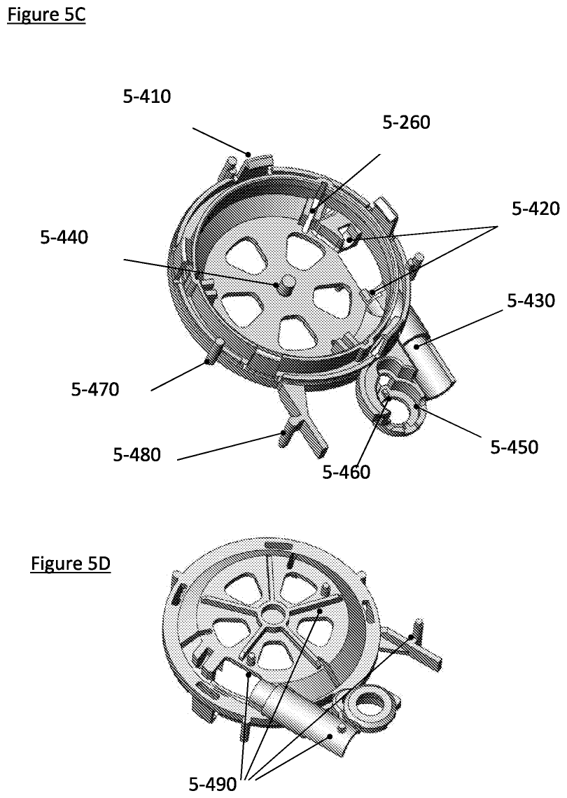

FIG. 4A provides a cut-away side view of an embodied prefilled selectively activatable infusion-pump assembly of this invention. In this figure, the outline of the pre-filled aseptically-sealed flexible drug reservoir-containing assembly, primary container 4-70 is marked, and needle insertion assembly 4-210 with needle 4-220 that opens the fluid path with respect to same is shown. Elements of the cannulated needle dispensing element 4-65 are shown. In this aspect, the cannula cap 4-60 covering the exterior of the undeployed cannulated needle dispensing element is shown. It is also evident from the figure that depressing the activator button 4-140 engages and rotates a switch 4-230, by means of the extension 4-145, which in turn may permit downstream events leading to the opening of the fluid path and insertion of the cannulated needle assembly in the skin of the subject. Same may also activate the PCB assembly 4-100 to power the motor (seen more clearly in FIG. 4B), engaging the worm drive, which in turn engages the lifting gear -4-190 propelling the piston 4-200 upward to impinge on the flexible pre-filled aseptically-sealed flexible drug reservoir-containing assembly, resulting in the pressurized release of drug contained therein, with the flattening of the reservoir as pressure is applied thereto from the lifting gear.

FIG. 4B and FIG. 4C provide greater detail highlighting the positioning of the Motor 4-245, which promotes rotation of the worm gear 4-250, which engages the lifting gear 4-190, which impinges on the flexible pre-filled aseptically-sealed flexible drug reservoir-containing assembly via the piston 4-200 promoting drug expulsion therefrom. The chassis 4-180 contains various structural accommodations to align and operationally connect the indicated parts, as well as providing a floating attachment for the worm gear 4-250 and motor 4-245, as further described. For example, referring to certain slots 4-260 in the chassis align with tabs 4-270 supported in part by ribs 4-275 on the piston, which prevents the piston from turning laterally and instead propels same upward, when engaged by the lifting gear 4-190 and the lead screw interface 4-280 of the lifting ear engages the threading hole 4-290 of the piston.

FIG. 4D depicts a basal view of an embodied piston component of an engine assembly as herein described. The piston will be operationally connected to the lifting gear, such that engagement of the worm gear, operationally connected to the motor results in engagement of the lifting gear and further engagement of the piston operationally connected thereto. FIGS. 5A and 5B provide a view of the engagement of the motor, worm gear and lifting gear, which when viewed in terms of FIGS. 4D, 4E and 4F, provide an understanding for the propulsion of the piston toward the flexible pre-filled aseptically-sealed flexible drug reservoir-containing assembly enabling drug delivery therefrom.

Referring to FIG. 4D and FIG. 4E, the operational connection between the lifting gear and piston may be accomplished via a specialized interface, for example, such as a modified screw interface 4-280 of the lifting gear 4-190, which engages a threaded region 4-290 on the piston such that rotation of the screw interface 4-280 of the lifting gear 4-190 promotes controlled upward propulsion (via e.g. unwinding) of the piston whose rotation is prevented by the tabs 4-270 on the piston being locked in place in slots 4-260 on the chassis.

FIG. 4F highlights key regions of the worm drive 4-250. The worm drive is operationally connected to a motor via a specialized region on the drive 4-340. In one aspect, the connection may be a D-shaped interface.

In some aspects, the worm drive connection to the motor via a specialized region that is D-shaped specifically connects to the motor shaft, which itself is D-shaped. According to this aspect, and in some embodiments, the D shape promotes transfer of rotational movement between parts without slippage.

In some aspects the worm gear engages and thereby rotates the lifting gear via specialized regions 4-320 on the worm gear and appropriately modified interacting components 4-195 on the lifting gear (FIG. 4E). In some aspects, the worm gear contains freely slidable surfaces 4-330, which allow for free axial movement of the worm gear within the chassis.

In some aspects, a reflecting surface 4-350 may be included.

According to this aspect, and in some embodiments, the proximal device surfaces to the worm gear apparatus will be “painted”, i.e. containing a non-reflective surface, and same facilitates counting the number of rotations of the gear about an axis during operation.

According to this aspect, and in some embodiments, the device further incorporates an optical sensor, positioned in the housing and located below the reflecting surface of the worm gear. Such sensor undergoes a change in signal as a consequence of reflection from the worm gear identified, as a function of reflection off the two reflective surfaces on the worm gear. According to this aspect, with each worm gear full rotation, two optical signals are relayed, and same in turn may be used to control piston speed and infusion rate and to indicate when the infusion has ended.

Thus, for example, referring to the flat region 4-350 described above, located on e.g. opposing outer sides of the worm gear, as depicted, if same are provided in a white color or reflective color, whereas the remaining elements of the worm gear are provided in an absorptive color, e.g. black, then the sensor in some embodiments, will detect a change in reflective signal, which in turn signifies partial rotation. In some aspects of the described herein, a full rotation of the worm drive may produce two “on” or reflective signals, thereby serving as an indicator for rotation of the worm gear.

Importantly, the thrust bearing surface 4-300 is disposed of distally form a floating motor shaft connection that is supported by the chassis rather than the motor shaft. The floating attachment combined with the placement of the thrust bearing surface 4-300 allows for enough radial displacement of the worm gear during operation as not to transmit damaging radial or longitudinal forces to the motor's planetary gear reduction system while ensuring there is minimal longitudinal play so as not to affect the precision of the drive gear rotation. The single thrust bearing surface 4-300 and floating attachment of the worm to the motor shaft 4-340 also allows for the simple alignment of the parts during assembly.

In some aspects, incorporation of simple DC motor is envisioned, whereby same is operationally coupled to the planetary and worm gear, as described, with no axial or radial load being applied to the motor. According to this aspect, and in some embodiments, such arrangement ensures transfer of torque only.

According to this aspect, and in some embodiments, upon electrical activation the motor, worm gear and lifting gear rotate, unscrewing the piston, and in some embodiments, the piston tabs mounted inside the chassis prevent the piston from rotating.

Further according to this aspect, and in some embodiments, there are then four mechanical interfaces within the motor-worm-chassis assembly, and as will be appreciated, the connection between the motor and chassis and worm gear and chassis at the distal end would contain tight connections between same, while the connection between the worm gear and motor and worm gear and chassis proximal to the motor shaft, would be a loose connection.

FIG. 5A depicts the motor 5-245 and worm drive 5-250 being mounted in a radial orientation to the chassis 5-360, representing some embodiments of the invention. It is noted that the connection between the motor 5-245 and the chassis 5-360 is a tight connection while the connection between the worm gear 5-250 and the chassis 5-360, as well as the connection between the worm gear 5-250 and the motor 5-245 are both loose connections, at the connection point proximal to the connection of the worm gear to the motor and worm gear to the chassis. The connection of the worm gear to the chassis is however a tight connection at the far end, distal to the connection point of the worm gear to the motor. It is noted that the far/distal end represents the thrust bearing surface.

Both the motor and worm gear, according to this aspect, are inserted sequentially in the chassis for ease of assembly. In some aspects, the worm gear is first inserted into the chassis (e.g. snapped in, using features as depicted by 5-300). According to this aspect, once the worm gear is positioned in place the motor is then inserted, for example, via inserting same in the same linear direction.

Moreover, in viewing FIGS. 5A and 5B, the attachment of the thrust bearing surface 5-300 to the chassis via a modified connection 5-390 and similarly providing a chassis connection to the motor gear 5-370 removes/reduces strain and promotes free axial movement 5-400 of the worm gear]

FIGS. 5C and 5D provide exploded views of some of embodied elements of the chassis. For example, slots 5-260 for the accommodation of the tabs on the piston, as described with respect to FIG. 4C and FIG. 4D are seen. A central pin 5-440 is also shown, which may facilitate centering of the lifting gear (see 4-190 in e.g. FIG. 4C or FIG. 4E). Sliding surfaces 5-420 for the accommodation of the worm gear and for the mounting of the motor 5-430 are shown, as well. Elements of the cannulated needle dispensing assembly traverse the chassis, as well, and a snap-to-fit element 5-460 on the chassis interfacing with same is shown, as is a mounting region 5-450 for the cannulated needle dispensing assembly to insert therethrough.

Similarly, various pins to facilitate alignment of other parts to promote proper fitting vis a vis the chassis and underlying PCB assembly, are shown. For example, referring to FIGS. 5C and 5D, a support or buttress 5-480 for battery elements connecting to the PCB assembly positioned underneath the chassis and traversing same may be desired. Similarly, the apically located flexible pre-filled aseptically-sealed flexible drug reservoir-containing assembly may be secured via snaps or fasteners 5-410 on the chassis, as well Still further, it may be desirable to incorporate additional pins 5-470 to properly align the chassis with a fitted mechanism on the internal apical housing cover and/or to incorporate additional pins 5-490 to properly align the chassis with a fitted mechanism on the basally located PCB assembly or further with the internal basal housing cover (not shown).

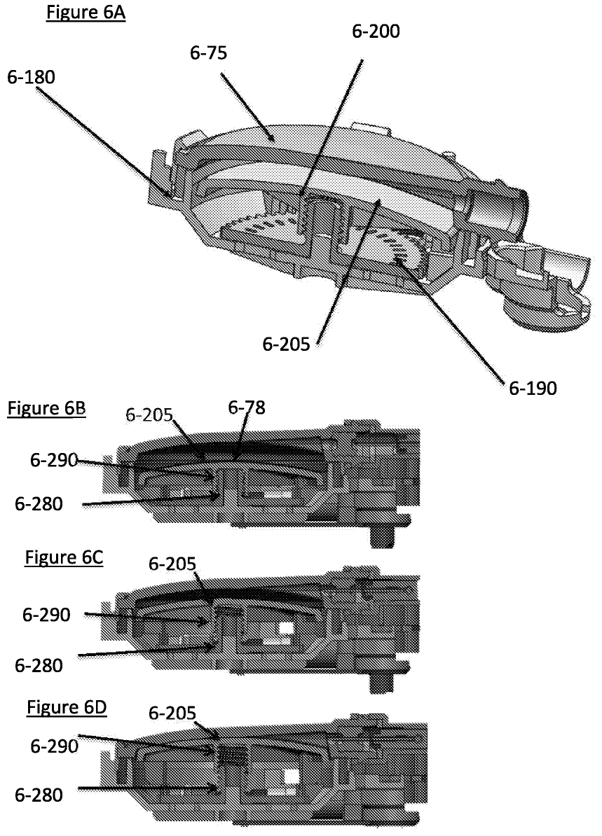

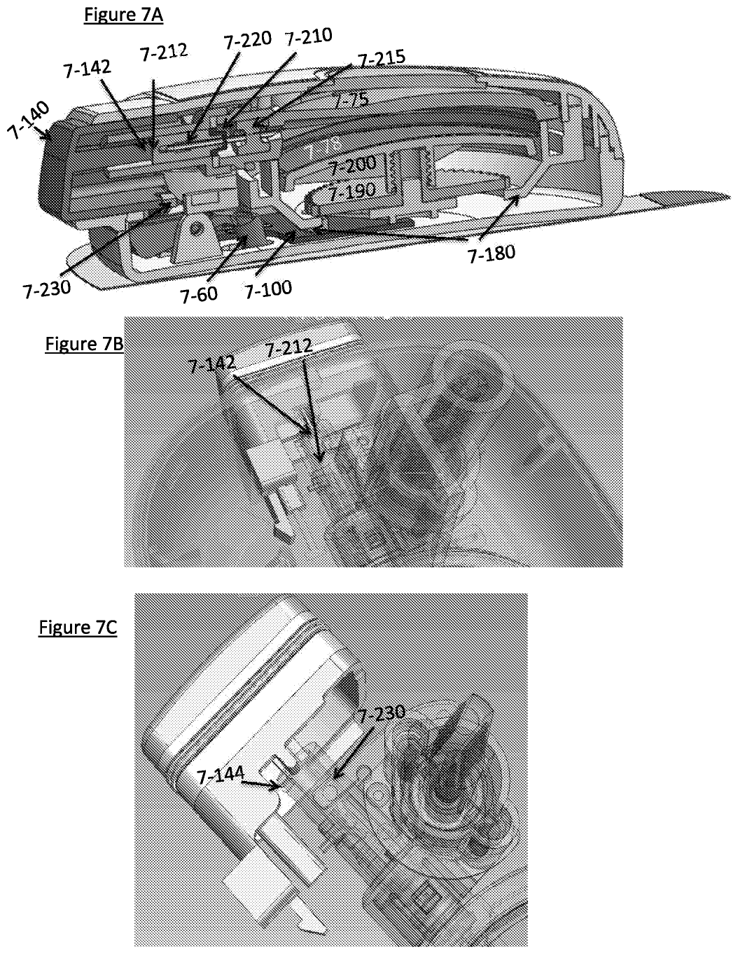

FIG. 6A depicts certain elements of an embodied prefilled selectively activatable infusion-pump assembly in non-deployed orientation. According to this aspect, as schematically depicted, the orientation of the chassis 6-180 with respect to the lifting gear 6-190 and piston 6-200 is seen. The piston top surface 6-205 is depicted, abutting the basal surface of the flexible pre-filled aseptically-sealed flexible drug reservoir-containing assembly (the basal surface is not shown) and the flexible pre-filled aseptically-sealed flexible drug reservoir-containing assembly is bounded by an upper wall 6-75 depicted in this figure.

As described herein, the motor is operationally connected to the worm gear, which in turn is operationally connected to the lifting gear, which in turn is operationally connected to the piston and this relay system promotes impingement of the piston on the basal surface of the flexible pre-filled aseptically-sealed flexible drug reservoir-containing assembly promoting drug egress therefrom. FIGS. 6B, 6C and 6D provide a schematic representation of the coordinated action of same.

Referring to FIG. 6A, the basal surface 6-78 of the flexible pre-filled aseptically-sealed flexible drug reservoir-containing assembly is seen and the apical surface 6-205 of the piston is shown in close proximity thereto. In accordance with this embodied aspect, a lead screw interface 6-280 of the lifting gear engages a threaded region 6-290 of the piston, and in the undeployed state, a tight fitting of the two is achieved, such that the top of the screw interface 6-280 of the lifting gear is essentially flush with a top region of the threaded area 6-290 of the piston. In FIGS. 6B, 6C and 6D, engagement of the worm gear and rotation of the lifting gear 5-190 thereby results in an unwinding of the lead screw interface 6-280 of the lifting gear and threaded region 6-290 of the piston. As the unwinding continues, there is almost a complete separation between the lead screw interface 6-280 of the lifting gear and threaded region 6-290 of the piston, resulting in a net upward movement of the apical surface of the piston 6-205. Thus, the apical surface of the piston 6-205, which initially is in close proximity to the basal surface 6-78 of the flexible pre-filled aseptically-sealed flexible drug reservoir-containing assembly continues to be moved upwards until it pushes the basal surface 6-78 of the flexible pre-filled aseptically-sealed flexible drug reservoir-containing assembly upward, as well, expelling the contents of the flexible pre-filled aseptically-sealed flexible drug reservoir-containing assembly.

The prefilled selectively activatable infusion-pump assembly will contain a conjoined, coordinately controlled and perpendicularly arranged cannulated needle dispensing and needle insertion assembly operationally connected to and in fluid connection with the pre-filled aseptically-sealed drug reservoir-containing assembly.

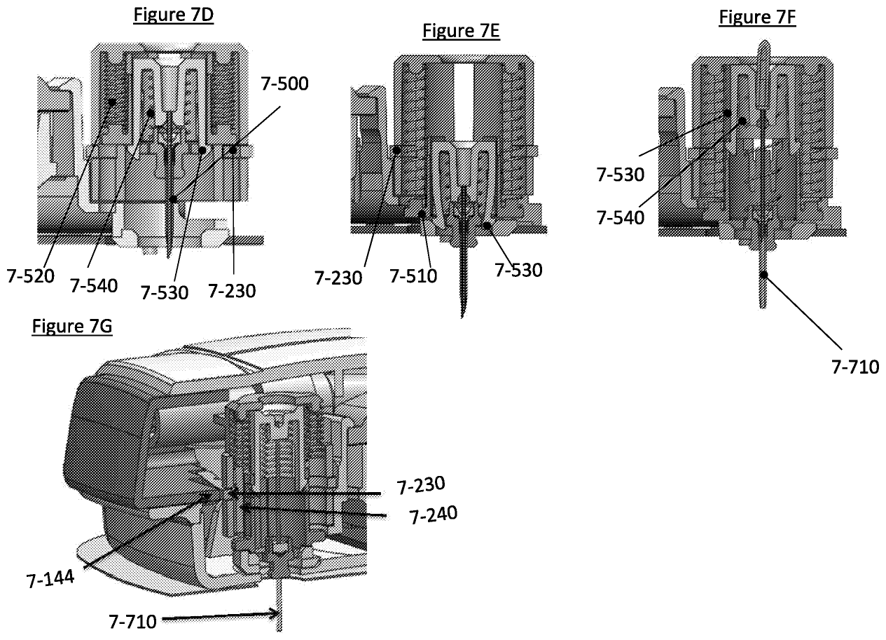

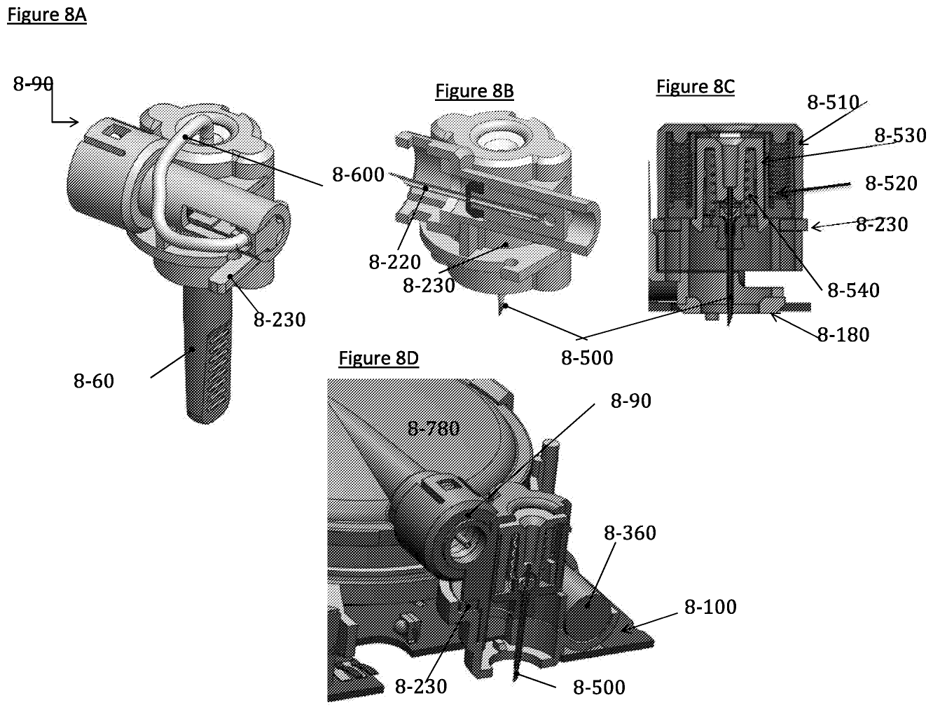

Referring to FIGS. 7A, 7C and 8A, various views of the elements of the coordinately controlled and perpendicularly arranged cannulated needle dispensing and needle insertion assembly and their operational and fluid connection to the pre-filled aseptically-sealed drug reservoir-containing assembly are shown.

As is also noted, the single step activator when engaged simultaneously promotes activation of the needle insertion assembly to open a fluid path with the pre-filled aseptically-sealed drug reservoir-containing assembly and activation of the cannulated needle dispensing assembly inserting in a skin of a subject.

In some embodiments, such single step activator may include an activator button modified to engage a number of other elements to achieve the coordinated controlled deployment of the different systems.

Referring to FIGS. 7A and 7C, the embodied device contains an activator button, which button is modified, in this aspect to differentially engage the cannulated needle dispensing and needle insertion assembly seen in FIG. 8A. In some aspects, the activator button 7-140 directly engages the needle insertion assembly 7-210, via engagement of a modified portion of the activator button 7-142 and a segment 7-212 of the needle insertion assembly, which in turn propels the needle 7-220 located therein to be propelled toward and ultimately pierce the septum 7-215, thereby opening a fluid path with the pre-filled aseptically-sealed drug reservoir-containing assembly (FIG. 7A). FIG. 7B provides a bottom 90 degree rotated view illustrating engagement of the adaptation on the activator button 7-142 with the segment of the needle insertion assembly 7-212. Depressing the activator button 7-140 in this aspect also promotes insertion of said cannulated needle in skin of a subject. Turning now to FIG. 7C, another modified adaptation on the activator button 7-144 may engage and move a “trigger” 7-230, which when thus engaged may move the trigger to release a blockade of the cannulated needle dispensing assembly. Ultimately, as is evident in this figure, the needle element retracts, allowing for insertion of the catheter component 7-710 within the skin of a subject.

FIG. 7D depicts the cannulated needle dispensing assembly when in the “storage condition”, i.e. prior to deployment. According to this aspect, the cannulated needle 7-500 is in its retracted position, and the needle hub 7-530 is in its locked position such that both sets of springs 7-520 and 7-540 controlling deployment of the cannulated needle dispensing assembly, including needle hub 7-530 and needle hub bushing 7-510 are in a compressed state and the trigger 7-230 is in its pre-activation position, as well.

FIG. 7E depicts initial insertion of the cannulated needle dispensing assembly within the skin of a subject, whereby the lateral rotation/movement of the trigger liberates the hub 7-530 and bushing 7-510 so that the bushing springs 7-520 expand and propel the cannulated needle in the needle hub downward. The hub 7-530 is ultimately from continued downward mobility by a fitting on the chassis that prevents continued downward deployment of same.

FIG. 7F depicts needle hub retraction, whereby the hub and needle retract by central spring retraction, pulling the needle hub upwards while leaving the cannula inserted within the skin of the subject.

FIG. 7G, similar to FIG. 7F depicts a rotated view illustrating engagement of the adaptation on the activator button 7-144 with the trigger 7-230.

FIGS. 8A-8D provide various embodied views of the coordinately controlled and perpendicularly arranged cannulated needle dispensing and needle insertion assemblies.

According to this aspect, elements of the needle insertion assembly 8-90 and cannulated needle dispensing assembly 8-500 is shown.

FIG. 8B depicts the needle insertion assembly, highlighting the positioning of the insertion needle 8-220 and positioning of the trigger 8-230. The dorsal positioning of the cannulated needle 8-500 is discerned in this figure, as well.

FIG. 8C depicts the elements of the cannulated needle dispensing assembly activated after rotation of the trigger 8-230. A core mechanism is released, whereby two bushing 8-510 springs 8-520 push the cannulated needle 8-500 into the skin of a subject. The needle hub 8-530 hits an extension/adaptation of the chassis 8-180 and the needle hub 8-530 retracts due to the retraction spring 8-540 force.

In some aspects, the printed circuit board (PCB) assembly 8-100 comprises metal parts or metal coated parts that come into contact e.g. with metal components of the activator assembly and complete a circuit when in contact therewith, such that the PCB assembly is thereby activated and promotes coordinate activation of the engine assembly operationally connected thereto.

FIG. 8D depicts the relative positioning of different elements of the prefilled selectively activatable infusion-pump assembly of this invention. In this figure the cannulated needle 8-500 is shown, as is the “trigger” 8-230, which allows for regulated movement of the cannulated needle as described. The needle insertion assembly 8-90 and its orientation with respect to the drug reservoir containing assembly 8-780 is shown.

Referring now to FIGS. 9A, 9B and 9C, there is provided a more detailed view of the regulated release of the cannulated needle dispensing assembly. In FIG. 9A, the trigger 9-230 has not yet been rotated by the activator and thus an adaptation 9-235 still somewhat blocks the needle bushing 9-510 deployment, by preventing passage of an extension 9-515 of the bushing from passing through the channel in the trigger 9-230 (compare FIGS. 9A versus 9B). While FIG. 9A provides a top view, FIG. 9B provides a bottom view.

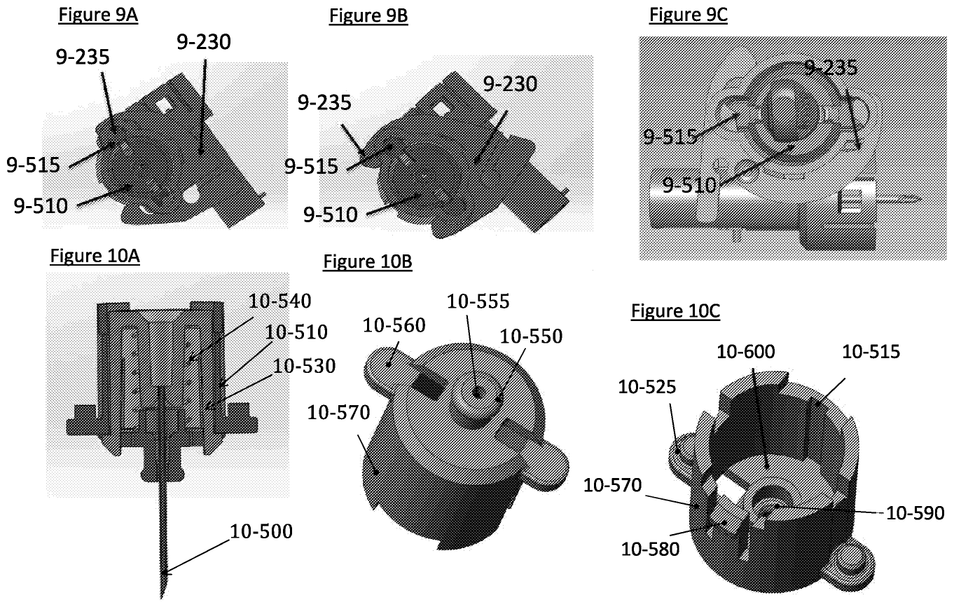

Referring to FIGS. 10A, 10B and 10C, a more detailed view of the cannulated needle dispensing assembly and its bushing is depicted. The cannulated needle dispensing assembly has, in addition to the needle 10-500, a specialized needle hub 10-530 is housed within a needle bushing 10-530. The needle is maintained in a retracted undeployed state ultimately propelled when deployed, with the aid of a needle spring 10-540 and then further retracted, once the cannula is inserted within the skin of the subject, as described herein.

FIGS. 10B and 10C provide greater detail regarding the needle bushing exterior and interior, respectively. The bushing appendage 10-550, which secures the cannulated needle inserting therethrough 10-555 is depicted. The bushing as part of the assembly descends and thus possesses a slidable surface 10-570 to facilitate same. The bushing also comprises a stopper for the needle hub 10-560. Additional modifications include slotting to prevent rotation of the needle hub when located within the bushing. Additional stabilizing structures on the bushing include snaps 10-580, which snaps to the cannula housing and prevents further movement of the bushing. The cannula septum seals within the bushing at part 10-590.

Spring structures are associated with the cannulated needle dispensing assembly to both propel the cannulated needle assembly downward toward the skin of a subject and to retract the needle after the cannula is stably inserted within the skin of the subject. The springs are referred to as insertion and retraction springs, respectively. The insertion springs attach to the needle bushing at a region so designed to accommodate same, for example as depicted by part 10-525 in FIG. 10C. The retraction spring similarly attaches at part 10-600 in the needle bushing as depicted in FIG. 10C.

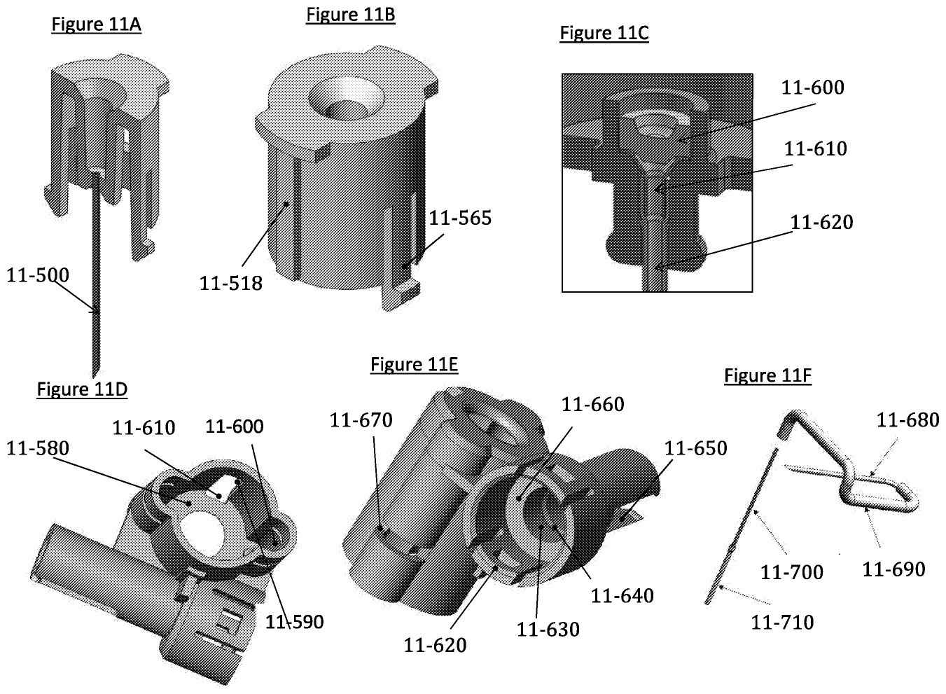

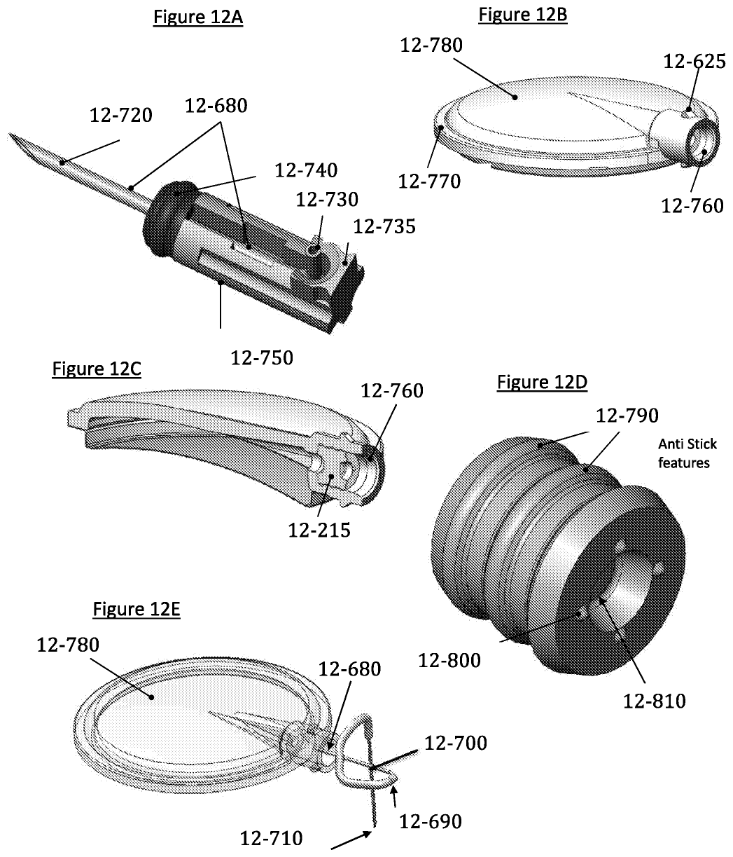

FIG. 11A and FIG. 11B provide greater detail regarding the needle hub. The (ultimately cannulated) needle 11-500 attachment to the hub 11-530 is shown. Referring to FIG. 10C and FIG. 11B, the needle hub contains an extension 11-518, which fits within the slot 10-515, which prevents movement/rotation of the needle hub. Other extensions, e.g. snaps such as 11-565 help affix the hub against the bushing, for example, affixing near part 10-560 on the bushing as shown in FIG. 10B.

FIG. 11C highlights the incorporation of, for example the cannulated assembly, through with the needle hub inserts. The cannulated assembly may comprise a septum 11-600, which until pierced promotes sterility of the assembly, as well. The cannulated assembly may also further comprise a funnel 11-610 and cannula 11-620.

FIG. 11D depicts elements of the conjoined, coordinately controlled and perpendicularly arranged cannulated needle dispensing and needle insertion assembly. In this aspect, the framework for the cannulated needle dispensing assembly is shown 11-240. The bushing is contained within the area 11-580, and a surface for affixing the bushing 11-610 and stop for the bushing locker/snap 11-590 is shown, as is the location for the insertion spring 11-600. The Sliding Surface for Insertion trigger 11-670 is also shown in FIG. 11E.

FIG. 11E is a rotated view of the conjoined, coordinately controlled and perpendicularly arranged cannulated needle dispensing and needle insertion assembly depicted in FIG. 11D, to further highlight the framework for the cannulated needle insertion assembly. The insertion mechanism is contained/attached to the drug reservoir baseplate 11-660, and a surface for centering same is shown at 11-620. Also depicted is the inner diameter 11-630 region relieving pressure in the assembly, and the surface to which the needle is affixed 11-640 and then slidably housed 11-650, which is depicted, as well.

FIG. 11F depicts further elements of the conjoined, coordinately controlled and perpendicularly arranged cannulated needle dispensing and needle insertion assembly. According to this aspect, the cannulated 11-710 dispensing needle 11-700 and insertion needle 11-680 are shown, as is the fluid connection between the two 11-690, via for example tubing interconnecting the two.

FIG. 12A provides a more detailed view of embodied insertion needle assembly elements. The needle insertion assembly projects generally in a parallel orientation to a generally planar surface of the housing, and the projection initiates opening a fluid path with the pre-filled aseptically-sealed drug reservoir-containing assembly. In some aspects, the pre-filled drug reservoir-containing assembly is aseptically sealed via the incorporation of a sterile septum, described further herein. In some aspects, opening the fluid path with the drug reservoir-containing assembly involves the insertion needle assembly promoting piercing of the septum. The insertion needle 12-680 may have a modified terminus 12-720 that is beveled and effectively pierces the septum or other closure of the drug reservoir-containing assembly. The insertion needle 12-680 is further modified at its distal terminus to interface with the connecting tubing, for example, 11-690 as seen in FIG. 11F. A sealing element 12-740 promotes further forming an effective seal between the drug reservoir-containing assembly (e.g. the baseplate) and the needle insertion assembly. The needle insertion assembly may contain further lateral modifications to slidably mount same, for example containing an extension 12-750, which could slidably be displaced within a grooved structure in the housing (e.g. 11-650 in FIG. 11E). Engagement with the distal terminus 12-735 in turn promotes lateral movement of the needle insertion assembly to project generally in a parallel orientation to a generally planar surface of said housing.

FIG. 12B depicts some elements of the pre-filled aseptically-sealed drug reservoir-containing assembly. In this aspect, the assembly may contain modified extensions 12-625 that insert within appropriately modified cognate surfaces in the housing, for example element 11-620 in FIG. 11F. The pre-filled aseptically-sealed drug reservoir-containing assembly 12-780 will contain a connecting neck 12-760 into which the needle insertion assembly inserts and is stably joined to create a aseptic fluid path. The pre-filled aseptically-sealed drug reservoir-containing assembly may contain flat lateral surfaces along the baseplate 12-770, that can be securably fastened to the chassis. FIGS. 12C and 12D show cross-sectional rotated views of the pre-filled aseptically-sealed drug reservoir-containing assembly. Shown is the septum 12-215, which promotes sealing of the drug reservoir, which is then pierced as described by the insertion needle assembly action.

FIG. 12D shows an enlarged view of an embodied septum, depicting the sealing ribs 12-790, and terminal modifications 12-800 to reduce sticking thereto.

In some aspects, certain components, e.g. the Chlorobutyl-containing components may become sticky after sterilization. According to this aspect, the embodied septum design ensures good separation between components and ease with handling and eventual assembly into the device.

In some aspects of the invention, the septum will contain a terminally recessed portion 12-810, which reduces the piercing force and needle deflection therethrough.

In one aspect, the terminally recessed portion will have a recess ranging from 0.1-5 mm.

In some aspects, the terminally recessed portion will have a recess of about 1 mm from each terminus of the septum. According to this aspect, and in some embodiments, the total septum length is 5 mm, further comprising 2 terminally recessed portions having a recess each of about 1 mm, and in some embodiments, therefore, the piercing length is about 3 mm.

FIG. 12E similarly depicts the conjoined, coordinately controlled and perpendicularly arranged cannulated needle dispensing and needle insertion assembly as shown in FIG. 11F, and its relative positioning in terms of the pre-filled aseptically-sealed drug reservoir-containing assembly, to facilitate opening the fluid path. Depicted in this embodied schematic is the cannulated 12-710 dispensing needle 12-700 and insertion needle 12-680 are shown, as is the fluid connection between the two 12-690, via for example, tubing interconnecting the two.

As described herein, it is a further objective of the present invention to provide the prefilled selectively activatable body-worn infusion-pump assembly comprising an aseptically pre-filled drug reservoir, a fluid connection means and a cannula insertion means where the assembly can be fitted to the rest of the infusion-pump components while maintaining sterility and requiring no actions on the part of the end user other than removing the cannula protection cap, adhering the assembled device onto the injection site and pressing a button.

According to this aspect, and in some embodiments, the devices of this invention comprise a single activator part, which is so designed to simultaneously or essentially simultaneously activate 3 steps of depressing a pre-filled drug reservoir to aid in delivery of the drug contained therein, open a fluid path to promote egress and delivery of the drug contained therein and pierce the skin of the user, to promote delivery of the drug contained therein to the user.

The prefilled selectively activatable infusion-pump assembly of this invention may comprise a further level of controlled delivery in the presence of a skin sensor operationally related to the activator, such that without appropriate detection of placement of the device on the skin of the user, the activator button is blocked and initiation of drug delivery from the assembly of the invention is prevented.

In some aspects, and representing embodied elements of the invention, the skin sensor assembly will comprise an optical sensor including a transmitter and receiver, a skin sensor pad with a mechanical barrier (also referred to herein as a “flag”) and a rocker and spring mechanism.

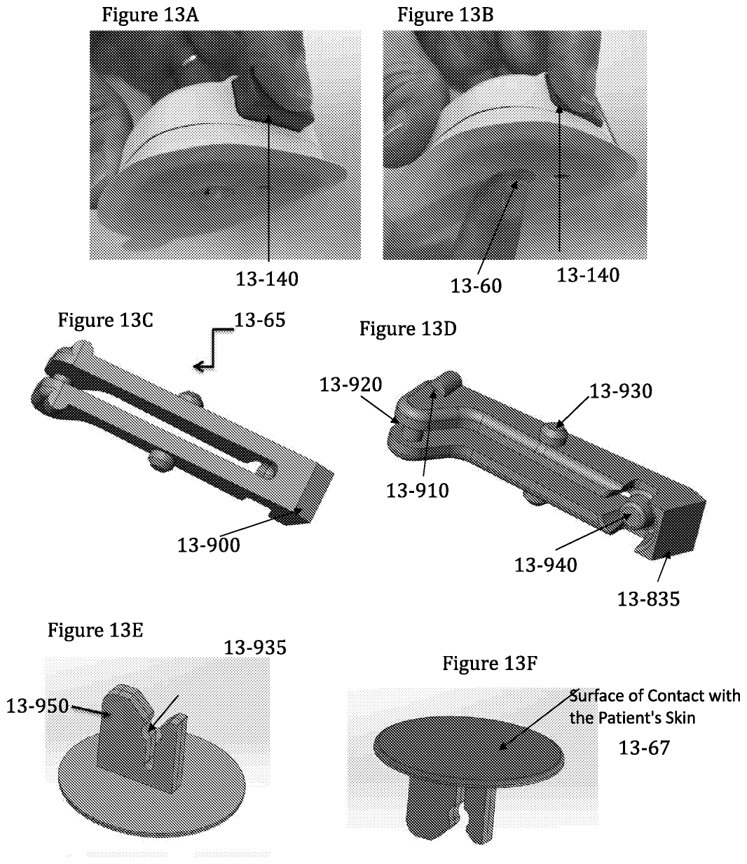

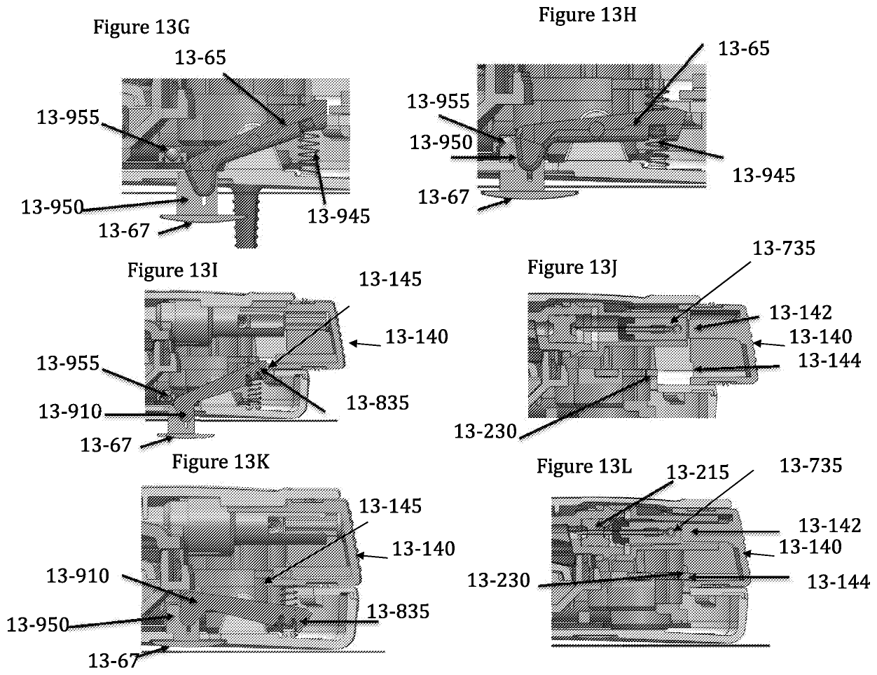

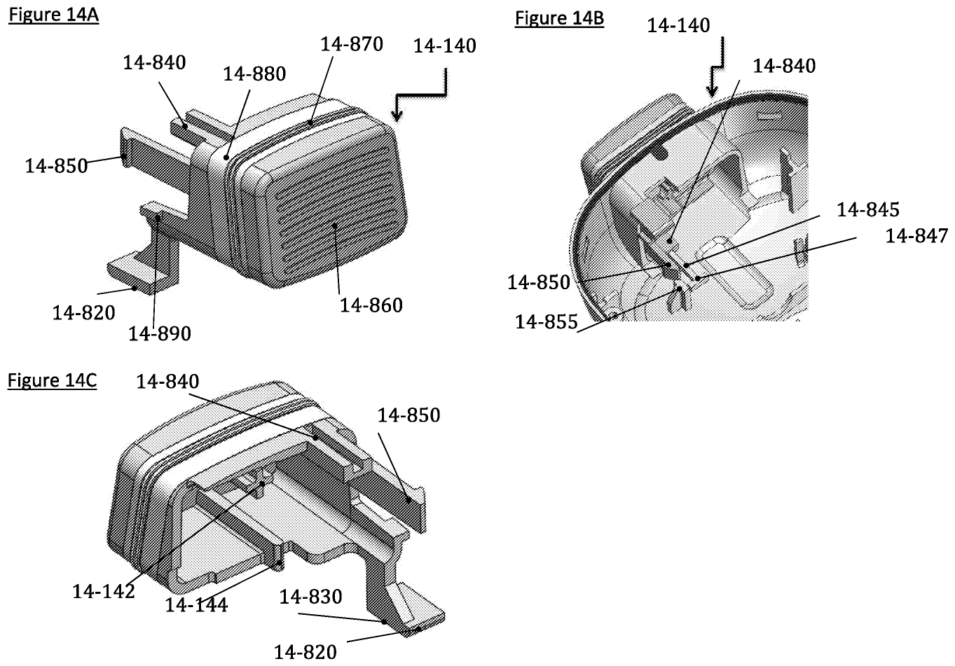

Referring for Example, to FIGS. 13A-13F, one embodied skin sensor relay regulatory component is shown. Referring to FIG. 13A versus FIG. 13B, where in FIG. 13B, skin contact with the sensor 13-60 is simulated, and depression of the activator 13-140 is achievable, whereas in FIG. 13A, no engagement of the sensor is shown and thus depression of the activator 13-140 is prevented.

Various configurations and parts for such a regulated engagement are envisioned. A non-limiting example of same may contain components as depicted in FIGS. 13C-F. In FIGS. 13C and 13D, the skin sensor support 13-65 is depicted, containing support surfaces during operation 13-900 and storage 13-910, respectively. The skin sensor is operationally connected to a spring, with the location of the connection to same 13-940 shown, which in turn promotes rotation of the support about an axis 13-930, to promote engagement of the sensor pad 13-67, in contact with the surface of a patient's skin and its sliding movement along the sensor pad axis of rotation 13-935, which rests on the support axis 13-920.

FIGS. 13E and 13F depict further elements of the skin sensor component including the presence of an opto block feature 13-950, which serves as a mechanical flag to indicate that the device has been removed from the body.

According to this aspect, and in some embodiments, the device further comprises an optical sensor on the PCB assembly, such that when the skin sensor is pressed on the skin of a subject, e.g. via the surface of contact with the patient's skin 13-67, the opto block feature is positioned such that the signal to the optical sensor on the PCB assembly is blocked, and thus, the remainder of events for initiating the device can proceed, since there has been proper indication that the pump is appropriately positioned on the body.

Upon disengagement of the skin sensor, the mechanical flag is rotated/removed, e.g. via the skin sensor pad axis of rotation 13-935 and this in turn prevents further delivery from the device.

Referring to FIG. 13G, 13H, 13I and 13K, the opto-block feature is further described.

In some aspects, prior to device activation, the skin sensor pad is in the “down” position, which is facilitated by force applied via spring 13-945 to the proximal end 13-835 of the rocker which pushes the proximal end of the rocker upward. In this orientation, the sensor relay system 13-955 is not obscured by the opto-block region 13-950 of the skin sensor.

According to this aspect, and referring now to FIG. 13K, after pump attachment to the skin, the skin presses against the pad, pushing the skin sensor pad 13-67 and attached flag 13-950 upward, which effectively blocks the optical path of the sensor 13-955 no longer evident in this view. Such blockade provides further feedback that engagement/activation of the apparatus may commence.

Referring to FIG. 13H, it will be appreciated that when the pump is being disengaged from a patient skin, either by design or as a result of malfunction, this will promote spring-activated ascent/ upward rotation of the rocker 13-65, which in turn promotes descent of the flag 13-950 and associated pad 13-67 promoting exposure of the optical sensor.

It will be appreciated that further regulation of this detachment phenomenon can be readily accomplished, for example, via SW algorithm that determines that the removal is intentional, for example after delivery ends and the user want to remove the device or if the pump has been detached unintentionally or due to malfunction, which, in turn may activate the sounding of an alarm or other warning notice in the device.

FIGS. 13J and 13L depict additional cross-sectional views of the device, where the relative positioning of the activation button 13-140 vis a vis the skin sensor parts reveals additional regulation preventing device activation unless the skin sensor is in the appropriate upright position, removing blockade of activation. Thus, comparing FIGS. 131 and 13J, the proximal end 13-835 of the rocker abusts the activation button extension 13-145, which physically prevents further depression of the button, such that another extension of the button 13-144 cannot engage the trigger 13-230. As is evident, the needle insertion apparatus is in the pre-activation condition, whereby the needle has not pierced the septum 13-215, and the fluid path has not yet been opened, therefore.

FIGS. 13K and 13L depict the skin sensor being located in its upright/ascended position, whereby the rocker rotation displaces the proximal end 13-835 of the rocker downward, clearing a path for the activation button extension 13-142 to engage the insertion mechanism 13-735 propelling the needle forward to pierce the septum 31-215; and as well displacing extension 13-144 to engage and laterally move the trigger 13-230, which as described, promotes activation of the cannulated needle assembly.

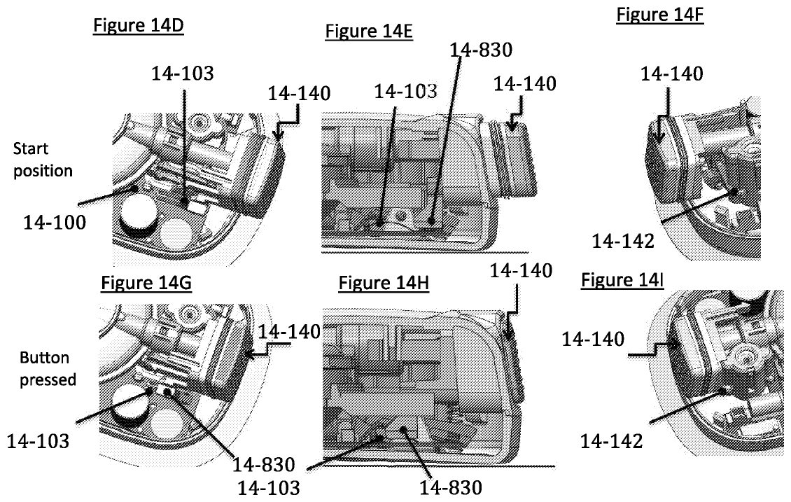

FIG. 14A provides a schematic depiction of an embodied activator 14-140. The activator may contain a push button surface 14-860, which is surface exposed, so that the user may in a single push step or graduated push step, depress the activator so that interfacing parts on the activator located internally to the device are engaged. In some aspects, the activator 14-140 will comprise sealing elements, such as sealing structures 14-880, and sealing ribs 14-870 form a tight seal with the device casing, maintaining a water-tight, air-tight seal.

The activator may contain further adaptations, such as locking mechanisms 14-850, which facilitate appropriate positioning and placement within the device. For example, and referring to FIG. 14B, the locking mechanism 14-850 snaps to fit within a defined cognate counter locking mechanism located on for example, a cover of the device, 14-855, which promotes a locking fit therein. The locking fit does not however prevent further advancement/sliding of the activator 14-140, as is appreciated by the skilled artisan, but does provide a framework for a regulated fit within the device.

In some aspects the activator may contain further adaptations, such as sliding guides 14-840, which promote proper propulsion/depression of the activator button so that interfacing parts on the activator located internally to the device are properly engaged. For example, and referring to FIG. 14B, the sliding guide may operationally interact with a slide 14-845 on the device cover, which allows for regulated advancement of the activator button along the track/slide thus defined. The slide may further contain a stopper 14-847, which stops further advancement in a defined manner.

In some aspects, the activator switch may contain further adaptations, such as, for example, mounting surfaces 13-890, which also facilitate proper positioning of the activator and its interfacing parts within the device.

FIG. 14C is a rotated view of the activator of FIG. 14A. In some aspects, the activator will comprise an adaptation 14-142, which serves as an interface promoting engagement with the needle insertion assembly.

FIG. 14C also depicts the electronic switch activation surface 14-830 which is located on the activation button 14-140, which when engaged promotes activation of the engine assembly contained in the housing operationally connected to said pre-filled aseptically-sealed flexible drug reservoir-containing assembly promoting depressing the pre-filled drug reservoir to promote drug delivery therefrom.

FIGS. 14D-14I provide additional detail of the activation button interface with an electronic switch on the PCB. Prior to engagement/depression of the activation button 14-140, as seen for example in FIGS. 14D-F, the electronic switch activation surface 14-830 is not in contact with the electronic switch 14-103 on the PCB. Comparing the undepressed versus depressed activation button position (FIGS. 14E versus 14H), the electronic switch activation surface 14-830 contacts and depresses the electronic switch 14-103, activating same, which in turn promotes powering of the worm gear and drug delivery apparatus, as described hereinabove.

Coincident with the activation of the electronic switch, as described above, the needle insertion assembly is propelled, which coordinately regulates/ensures opening of the fluid path and activation of the cannulated needle assembly.

In some aspects, the activator adaptations may be so constructed to allow regulation of steps, whereby the needle dispensing mechanism deployment occurs slightly later than the activation of the engine assembly and/or needle insertion assembly. In some embodiments, the activation of the engine assembly and/or needle insertion assembly is essentially instantaneous and other key steps in the drug delivery pathway are coordinately controlled to occur almost instantaneously, but a slight lag in time may occur and same does not interfere with appropriate controlled drug delivery achievable via the devices and methods of this invention.

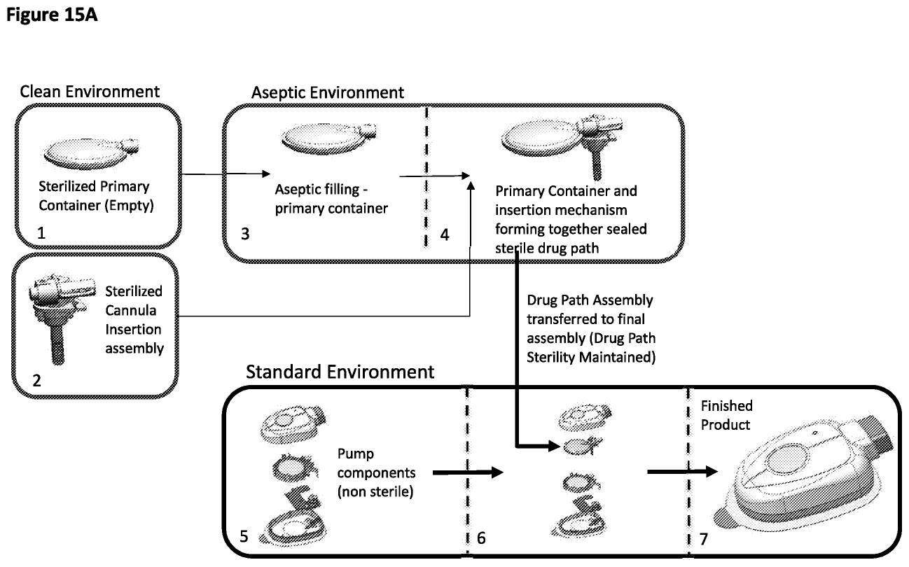

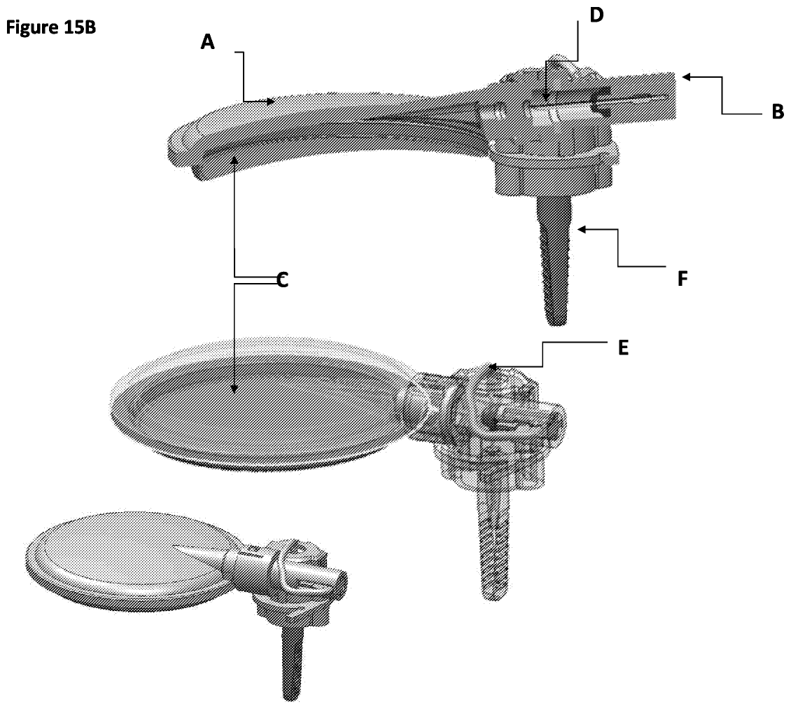

FIG. 15A-B depicts a flow chart of specific combinations of steps that promotes preparation of a sterilized drug reservoir that is aseptically filled, then connected to a pre-sterilized drug path connection system and cannula insertion system and finally assembled into the functional patch pump device such that the end user needs only to expose an adhesive surface, remove the cannula cap, adhere the device to the injection site and press a button in order to complete the injection process.

Such arrangement/assembly provides uniquely the ability to allow for an aseptically pre-filled container to be used with a skin-adhered patch pump, in marked contrast to known similar drug delivery devices currently in use, which require the end user, or an assistant to fill the pump reservoir and program the pump to deliver the appropriate dosage. Consequently, sterility of the drug path cannot be guaranteed as the path and drug solution is manipulated in a non-aseptic environment. Uniquely the subject invention promotes arrival at an economically viable patch-pump device that comprises the pre-filled drug reservoir and the complete sterile drug path in an economical disposable device that requires minimal manipulation by the end user.

In some aspects of the invention, prefilled injectable devices require a sterile drug path maintained during manufacturing, assembly and storage until the user's point of use.

In some aspects, advantages of the devices of this invention include the ability to prepare sterile drug path components such as a drug reservoir container and cannula insertion assembly, sterilized independently (see steps (1) and (2) in FIG. 15A), which can be packaged shipped and ultimately assembled in an aseptic environment.

For example, both components may be removed from their sterile barrier packaging inside an aseptic environment. Then, the drug reservoir container may be filled and capped with a septum (see step (3) in FIG. 15A) and afterward fitted with a sealed interface onto the cannula insertion mechanism (see step (4) in FIG. 15A).

The final assembly process (see for example, steps (5) and (6) of FIG. 15A) may be accomplished in a standard environment, i.e. full sterile assembly conditions are not required, since the device components which contain the drug and other parts that come into contact with the body are sterile, fully sealed and already assembled as part of this modular system. The remaining device components and final assembly is accomplished, packaged and stored until final use (see step (7) of FIG. 15A).

As will be appreciated by the skilled artisan, it is beneficial to require assembly of a more minimal number of parts in a fully sterile environment, as opposed to needing full assembly of all or most of the components of the device in a fully sterile environment.

FIG. 15B provides an enlarged and somewhat more detailed view of the device elements referred to in step 4 of FIG. 15A. As is apparent from this depicted aspect, there is a sterile sealing interface between the drug reservoir container (A) and Cannula insertion mechanism (B). The drug is stored inside the reservoir container (C). A septum must be pierced to gain access to the drug within the drug reservoir container, which in turn can be accomplished by means of the insertion needle (D). Both elements as noted are assembled in an aseptic environment using fully sterile components. Step E depicts the sterile fluid path connection between the needle insertion assembly and cannulated needle dispensing assembly, which is also assembled in asceptic conditions with fully sterile components, and a sterile cap (F) maintains a sterile barrier to the outside environment.

In some aspects, the assembly is such so as to ensure that sterility of the drug delivery path is maintained and no subsequent end-user stage assembly is required. In some aspects, the additional components, such as the engine assembly, housing and other parts are so joined so as to ensure that sterility of the drug path components is not compromised.

It will be understood by those skilled in the art that various changes in form and details may be made therein without departing from the spirit and scope of the invention as set forth in the appended claims. Those skilled in the art will recognize, or be able to ascertain using no more than routine experimentation, many equivalents to the specific embodiments of the invention described herein. Such equivalents are intended to be encompassed in the scope of the claims.

In the claims articles such as “a,”, “an” and “the” mean one or more than one unless indicated to the contrary or otherwise evident from the context. Claims or descriptions that include “or” or “and/or” between members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides, in various embodiments, all variations, combinations, and permutations in which one or more limitations, elements, clauses, descriptive terms, etc., from one or more of the listed claims is introduced into another claim dependent on the same base claim unless otherwise indicated or unless it would be evident to one of ordinary skill in the art that a contradiction or inconsistency would arise. Where elements are presented as lists, e.g. in Markush group format or the like, it is to be understood that each subgroup of the elements is also disclosed, and any element(s) can be removed from the group.

It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. Certain claims are presented in dependent form for the sake of convenience, but Applicant reserves the right to rewrite any dependent claim in independent format to include the elements or limitations of the independent claim and any other claim(s) on which such claim depends, and such rewritten claim is to be considered equivalent in all respects to the dependent claim in whatever form it is in (either amended or unamended) prior to being rewritten in independent format.

1.-41. (canceled)

42. A prefilled selectively activatable infusion-pump assembly comprising:
a housing;
a pre-filled aseptically-sealed flexible drug reservoir-containing assembly;
a conjoined, coordinately controlled and perpendicularly arranged cannulated needle dispensing and needle insertion assembly operationally connected to and in fluid connection with said pre-filled aseptically-sealed drug reservoir-containing assembly, wherein said cannulated needle dispensing assembly projects generally perpendicularly to a generally planar surface of said housing, promotes insertion of said cannulated needle in skin of a subject and promotes retraction of said needle within said cannulated needle dispensing assembly thereafter, and
wherein said needle insertion assembly projects generally in a parallel orientation to a generally planar surface of said housing, which projection initiates opening a fluid path with said pre-filled aseptically-sealed drug reservoir-containing assembly;
an engine assembly contained in said housing operationally connected to said pre-filled aseptically-sealed flexible drug reservoir-containing assembly promoting release of a drug contained therein, wherein said engine assembly comprises:
a motor;
a worm gear, operationally connected to said motor;
a lifting gear, operationally connected to said worm gear;
a piston operationally connected to said lifting gear; and
a chassis fitted with an attachment promoting a floating connection thereto with said worm gear;
wherein said worm gear and motor are mounted radially with respect to said chassis;
a printed circuit board (PCB) assembly, which mechanically supports, electrically connects and controls the function of at least said engine assembly; and
a single step activator that when engaged simultaneously promotes:
activation of said needle insertion assembly opening a fluid path with said pre-filled aseptically-sealed drug reservoir-containing assembly;
activation of said engine assembly; and
activation of said cannulated needle dispensing assembly inserting in a skin of a subject.

43. The prefilled selectively activatable infusion-pump assembly of claim 42, further comprising an indicator light or sound relay, an observation window, at least one basal adhesive panel, a skin sensor, a cannula cap, or any combination thereof.

44. The prefilled selectively activatable infusion-pump assembly of claim 42, wherein said single step activator comprises a laterally moving part operationally connected thereto, whose lateral movement removes a blockade of said needle insertion assembly opening a fluid path with said pre-filled aseptically-sealed drug reservoir-containing assembly; said cannulated needle dispensing assembly inserting in a skin of a subject, or a combination thereof

45. The prefilled selectively activatable infusion-pump assembly of claim 42, wherein said single step activator comprises a movable element such that said engine assembly is engaged upon deployment of said single step activator, or
wherein said single step activator comprises an element engaging an electronic switch activation surface of said engine assembly, powering same, or
wherein said single step activator comprises an element engaging an element which promotes closure of a circuit on said PCB assembly thereby activating said engine assembly, or
wherein said single step activator cannot be engaged or deployed as long as a skin sensor device detects that said prefilled selectively activatable infusion-pump assembly is not properly positioned on a skin of a subject.

46. The prefilled selectively activatable infusion-pump assembly of claim 42, wherein said conjoined, coordinately controlled and perpendicularly arranged cannulated needle dispensing and needle insertion assembly are arranged in an orientation that is substantially perpendicular with respect to each other.

47. The prefilled selectively activatable infusion-pump assembly of claim 42, wherein said cannulated needle dispensing assembly promotes insertion of a cannulated needle into a skin of a subject and promotes retraction of said needle within said cannulated needle dispensing assembly thereafter,
wherein said cannulated needle dispensing assembly comprises a first spring, retractable within said assembly, which when released from a compressed state propels said insertion needle downward and a second differentially addressable spring, retractable within said assembly, which when released from a compressed state propels said cannula downward, and
wherein said first spring is separately retractable after deployment from said second spring.

48. The prefilled selectively activatable infusion-pump assembly of claim 42, wherein said needle insertion assembly pierces a septum comprising ribbing on an outer surface of said septum, protrusions around a bore of either face of said septum, or any combination thereof.

49. The prefilled selectively activatable infusion-pump assembly of claim 42, wherein said worm gear comprises a thrust bearing surface disposed of distally from a floating motor shaft connection that is supported by the chassis rather than the motor shaft.

50. A prefilled selectively activatable infusion-pump assembly comprising:
a housing;
a pre-filled aseptically-sealed flexible drug reservoir-containing assembly;
a conjoined, coordinately controlled and perpendicularly arranged cannulated needle dispensing and needle insertion assembly operationally connected to and in fluid connection with said pre-filled aseptically-sealed drug reservoir-containing assembly,
said cannulated needle dispensing assembly comprising:
a first needle hub for securing said dispensing needle and associated first spring for deploying and retracting said dispensing needle; and
a second outer hub for securing said cannula and associated second spring for deploying said cannula;
wherein said second outer hub and first needle hub are in concentric arrangement and said first spring and second spring are separately addressable and retractable within said assembly, and wherein said first spring and said second spring when released from a compressed state propel said insertion needle and said cannula downward, respectively;
wherein said cannulated needle dispensing assembly projects generally perpendicularly to a generally planar surface of said housing, promotes insertion of said cannulated needle in skin of a subject and promotes retraction of said needle within said cannulated needle dispensing assembly thereafter, and
wherein said needle insertion assembly projects generally in a parallel orientation to a generally planar surface of said housing, which projection initiates opening a fluid path with said pre-filled aseptically-sealed drug reservoir-containing assembly;
a power source powering drug delivery; and
a single step activator that when engaged simultaneously promotes:
activation of said needle insertion assembly opening a fluid path with said pre-filled aseptically-sealed drug reservoir-containing assembly; and
activation of said cannulated needle dispensing assembly inserting in a skin of a subject.

51. The prefilled selectively activatable infusion-pump assembly of claim 50, wherein said conjoined, coordinately controlled and perpendicularly arranged cannulated needle dispensing and needle insertion assembly are arranged in an orientation that is substantially perpendicular with respect to each other.

52. The prefilled selectively activatable infusion-pump assembly of claim Error! Reference source not found., wherein said first spring is separately retractable after deployment of said second spring.

53. The prefilled selectively activatable infusion-pump assembly of claim 50, further comprising:
an engine assembly contained in said housing operationally connected to said pre-filled aseptically-sealed flexible drug reservoir-containing assembly promoting release of a drug contained therein, wherein said engine assembly comprises:
a motor;
a worm gear, operationally connected to said motor;
a lifting gear, operationally connected to said worm gear;
a piston operationally connected to said lifting gear; and
a chassis fitted with an attachment promoting a floating connection thereto with said worm gear;
wherein said worm gear and motor are mounted radially with respect to said chassis; and
a printed circuit board (PCB) assembly, which mechanically supports, electrically connects and controls the function of at least said engine assembly;
wherein said single step activator when engaged further simultaneously promotes activation of said engine assembly.

54. The prefilled selectively activatable infusion-pump assembly of claim 53, wherein said single step activator comprises an element engaging an element which promotes closure of a circuit on said PCB assembly thereby activating said engine assembly, or
wherein said single step activator comprises a movable element such that said engine assembly is engaged upon deployment of said single step activator, or
wherein said single step activator comprises an element engaging an electronic switch activation surface of said engine assembly, powering same.

55. The prefilled selectively activatable infusion-pump assembly of claim 50, further comprising an indicator light or sound relay, an observation window, at least one basal adhesive panel, a skin sensor, a cannula cap, or any combination thereof.

56. The prefilled selectively activatable infusion-pump assembly of claim 50, wherein said single step activator comprises a laterally moving part operationally connected thereto, whose lateral movement removes a blockade of said needle insertion assembly opening a fluid path with said pre-filled aseptically-sealed drug reservoir-containing assembly; said cannulated needle dispensing assembly inserting in a skin of a subject, or a combination thereof.

57. The prefilled selectively activatable infusion-pump assembly of claim 50, wherein said single step activator cannot be engaged or deployed as long as a skin sensor device detects that said prefilled selectively activatable infusion-pump assembly is not properly positioned on a skin of a subject.

58. The prefilled selectively activatable infusion-pump assembly of claim 50, wherein said needle insertion assembly pierces a septum comprising ribbing on an outer surface of said septum, protrusions around a bore of either face of said septum, or any combination thereof.

59. The prefilled selectively activatable infusion-pump assembly of claim 50, wherein said worm gear comprises a thrust bearing surface disposed of distally from a floating motor shaft connection that is supported by the chassis rather than the motor shaft.

60. A prefilled selectively activatable infusion-pump assembly comprising:
a housing;
a pre-filled aseptically-sealed flexible drug reservoir-containing assembly;
a cannulated needle dispensing assembly;
a needle insertion assembly operationally connected to and in fluid connection with said pre-filled aseptically-sealed drug reservoir-containing assembly;
an engine assembly contained in said housing operationally connected to said pre-filled aseptically-sealed flexible drug reservoir-containing assembly promoting release of a drug contained therein, wherein said engine assembly comprises:
a motor;
a worm gear, operationally connected to said motor;
a lifting gear, operationally connected to said worm gear;
a piston operationally connected to said lifting gear; and
a chassis fitted with an attachment promoting a floating connection thereto with said worm gear;
wherein said worm gear and motor are mounted radially with respect to said chassis; and
a printed circuit board (PCB) assembly, which mechanically supports, electrically connects and controls the function of at least said engine assembly.

61. The prefilled selectively activatable infusion-pump assembly of claim 60, wherein said worm gear comprises a thrust bearing surface disposed of distally from a floating motor shaft connection that is supported by the chassis rather than the motor shaft.

62. The prefilled selectively activatable infusion-pump assembly of claim 60, further comprising an indicator light or sound relay, an observation window, at least one basal adhesive panel, a skin sensor, a cannula cap, or any combination thereof.

63. The prefilled selectively activatable infusion-pump assembly of claim 60, wherein said assembly further comprises a single step activator that when engaged simultaneously promotes:
activation of said needle insertion assembly opening a fluid path with said pre-filled aseptically-sealed drug reservoir-containing assembly;
activation of said engine assembly; and
activation of said cannulated needle dispensing assembly inserting in a skin of a subject.

64. The prefilled selectively activatable infusion-pump assembly of claim 63, wherein said single step activator comprises a laterally moving part operationally connected thereto, whose lateral movement removes a blockade of said needle insertion assembly opening a fluid path with said pre-filled aseptically-sealed drug reservoir-containing assembly; said cannulated needle dispensing assembly inserting in a skin of a subject, or a combination thereof.

65. The prefilled selectively activatable infusion-pump assembly of claim 63, wherein said single step activator comprises a movable element such that said engine assembly is engaged upon deployment of said single step activator, or
wherein said single step activator comprises an element engaging an electronic switch activation surface of said engine assembly, powering same, or
wherein said single step activator comprises an element engaging an element which promotes closure of a circuit on said PCB assembly thereby activating said engine assembly.

66. The prefilled selectively activatable infusion-pump assembly of claim 60, wherein said single step activator cannot be engaged or deployed as long as a skin sensor device detects that said prefilled selectively activatable infusion-pump assembly is not properly positioned on a skin of a subject.

67. The prefilled selectively activatable infusion-pump assembly of claim 60, wherein said cannulated needle dispensing and needle insertion assembly are conjoined, coordinately controlled and perpendicularly arranged, or
wherein said cannulated needle dispensing assembly promotes insertion of a cannulated needle into a skin of a subject and promotes retraction of said needle within said cannulated needle dispensing assembly thereafter, or
wherein said cannulated needle dispensing assembly comprises concentric arrangement of a needle hub securing said insertion needle and a bushing separately securing said cannula in said assembly.

68. The prefilled selectively activatable infusion-pump assembly of claim 67, wherein said cannulated needle dispensing assembly comprises a first spring, retractable within said assembly, which when released from a compressed state propels said insertion needle downward and a second differentially addressable spring, retractable within said assembly, which when released from a compressed state propels said cannula downward,
wherein said first spring is separately retractable after deployment from said second spring.

69. The prefilled selectively activatable infusion-pump assembly of claim 60, wherein said needle insertion assembly pierces a septum comprising ribbing on an outer surface of said septum, protrusions around a bore of either face of said septum, or any combination thereof.
